(19) United States
(12) Patent Application Publication
(10) Pub. No.: US 2023/0225726 A1
(43) Pub. Date: Jul. 20, 2023

(54) SURGICAL SUTURING DEVICE

(71) Applicant: Via Surgical Ltd., Moshav Amirim (IL)

(72) Inventors: Ofek Levin, Moshav Amirim (IL); Arie Levy, Shoham (IL); Lena Levin, Moshav Amirim (IL); Tom Assaf, Haifa (IL)

(21) Appl. No.: 18/096,323

(22) Filed: Jan. 12, 2023

Related U.S. Application Data
(60) Provisional application No. 63/300,363, filed on Jan. 18, 2022.

(51) Int. Cl. A61B 17/04; A61B 17/06

(52) U.S. Cl. CPC A61B 17/0482; A61B 17/0469; A61B 17/06066; A61B 2017/00862

(57) ABSTRACT

The invention provides a device for delivering surgical sutures. Significantly, the device is capable of delivering sutures of any size stitch width or stitch length. For minimally invasive surgeries such as laparoscopic surgery or endoscopic surgery, the device allows for delivering sutures of a stitch width and depth that is not constrained by the diameter of the shaft of the instrument. Further, the device provides flexibility that improves the maneuverability and degrees of freedom in the range of motion in difficult-to-reach surgical sites. For example, devices of the invention provide for delivering sutures at an angle substantially offset from the axis of the instrument. This flexibility contributes to providing more secure sutures at a range of challenging surgical sites.

### FIELD OF INVENTION

The invention generally relates to devices for delivering surgical sutures.

### BACKGROUND

Various suturing procedures may be performed via minimally-invasive surgery, using laparoscopic or endoscopic tools. In such procedures, a small incision (e.g., no more than a few cm) may be made through a patient's skin. A tool called a trocar may be placed within the incision and a cannula extending through the trocar provides an open bore, e.g., of about 15 mm diameter, for access to the surgical site by endoscopic tools. Surgical instruments as well as an endoscopic camera may be inserted through such cannulas and used for surgical procedures such as wound closure, tissue repair, and fixation of prosthetics.

However, minimally invasive surgeries present some technical challenges. The limited dimensions of the cannula restrict access to the surgical site, resulting in a restriction in maneuverability and loss of degrees of freedom in the range of motion of the instruments. For example, due to the complexities of stitching and tying sutures in such restricted environments, some tools use pre-formed clips or fasteners. But a common problem is that such fasteners can be no larger than the cannula diameter, which results in limits to the amount of tissue that can be gripped or fastened by any single fastener. Additionally, pre-formed polymeric surgical clips are limited by their mechanical simplicity, as they commonly are molded, self-fastening clips or simple barbed or threaded tacks. Such pieces must be attached according to the shape, size, and mechanical disposition inherent in their design.

### SUMMARY

The invention provides a device for delivering sutures during minimally invasive surgeries in which stitching dimensions are not restricted by an instrument diameter confined to fit through a conventional cannula. Because the sutures are thread, braided, or monofilament, a surgeon has the full flexibility to tie off tissue and prosthetics in any shape and dimensions that the flexibility of thread permits. Delivery devices of the invention are engineered with a geometry that provides for stitching sutures in ways not limited by common features of the minimally invasive surgical approaches. Devices of the invention include an extended shaft that can be inserted through a trocar and reach to tissue within the patient, e.g., reach a length of 10 cm or further through a trocar with a 15 mm diameter. Embodiments use a deflectable member, such as a shelf or foot that folds radially away from the extended shaft once the device is inserted, to allow for delivery of stitches to locations that are off-axis from the delivery shaft. Significantly, devices of the invention are capable of delivering sutures to arbitrary stitch widths or lengths not limited by the diameter of the delivery shaft.

Various embodiments also provide a detachable member shaped as a loop, hook, clip, tab, catch, or loop piece that attaches to an end of a suture and is used by the suturing device to catch and hold the distal end of the suture in a position while the delivery mechanism retracts, in which, when the device is pulled away, the detachable loop is detached from the suture and carried away with the device leaving only the suture end extending through the tissue. For minimally invasive surgeries such as laparoscopic surgery or endoscopic surgery, the device allows for delivering sutures to a stitch dimension not constrained by the diameter of the shaft of the instrument. The device may be used to suture any type of anatomical tissue for any type of anatomical surgery such as organ or tissue repair. The device may be used for open surgery or minimally invasive surgeries, including laparoscopic or endoscopic surgeries, and may be used with a variety of common cannulas and trocars to access any variety of surgical sites or tissue types. The device is capable of adapting to and adjusting to different tissue surfaces and incision sites for a variety of stitch widths to meet surgical suturing needs. Further, the device provides flexibility that improves the maneuverability and degrees of freedom in the range of motion in difficult-to-reach surgical sites. For example, devices of the invention provide for delivering sutures positioned on tissue substantially offset from the axis of the instrument. This flexibility contributes to providing more secure sutures at a range of challenging surgical sites.

In one aspect, devices of the invention comprise a body with a handle, an elongated shaft extending from the body, a deflectable member coupled to the distal end of the shaft, a needle housed within the shaft, and a suture attached on the needle. The deflectable member is moveable between at least a first position extending along an axis of the shaft and a second position deflected away from the axis of the shaft. The needle housed within the shaft is in a substantially straight configuration and comprises a shape memory material that, when the needle is advanced out of the shaft with the deflectable member in the second position, gives the needle a curved shape that carries the tip of the needle to a location on the deflectable member spaced away from the axis of the shaft. The tip of the needle carries a first end of the suture on the needle to a location on the deflectable member spaced away from the axis of the shaft.

In embodiments, the deflectable member of the device is elastically biased to the first position. Preferably, the deflectable member comprises a tissue-facing surface. When the device is pushed against the surface of the tissue, the deflectable member deflects to the second position with the tissue-facing surface facing the surface of the tissue, even with the tissue face substantially normal to the axis of the shaft.

The delivery tip, e.g., a distal portion of the shaft, may be articulated, allowing the surgeon to orient the delivery tip suitable for an optimal approach to the target. For example, the shaft may have a living hinge or joint some distance proximal to the terminus (e.g., a few centimeters). The joint can be controllable from the handle by, for example, rotating a thumbwheel or lever on the handle to pull via a cable through the shaft pulls the distal delivery tip to an off-axis delivery position. This articulation may be in addition to the deflectable member. In fact, the combination of the hinge or joint plus the deflectable member creates a form of double jointed shaft in which the distal segment can bend away from the proximal and then the deflectable member and deflect away from an axis of the distal segment.

As with the distal segment, the deflectable member may be controllable from the handle by, for example, rotating a thumbwheel or lever on the handle to pull via a cable through the shaft to cause the deflectable member to deflect into a desired angle with the shaft axis.

The device may be used to deliver the suture into, and back out of, the tissue to leave the suture extending within the tissue with the first end emerging from the tissue at an exit point and the second end emerging from the tissue at an entry point. The entry point and exit point define a stitch length and the stitch length is substantially larger than the diameter of the shaft of the device. In devices of the invention, the needle passes the suture through tissue and leaves the suture in the tissue with first and second ends of the suture extending out of the tissue at respective first and second exit locations spaced apart a length on the surface of the tissue.

In embodiments, the deflectable member receives the first end of the suture from the needle and holds the first end of the suture when the needle retracts into the shaft. In other embodiments the first end of the suture comprises a loop or crook that is caught by the deflectable member when the needle is advanced out of the shaft. In devices of the invention, the deflectable member catches the first end of the suture when the needle is advanced out of the shaft to form the suture into a stitch in the tissue. Here, the first end of the stitch is initially made substantially along the axis of the shaft, and the second end of the stitch is initially made substantially displaced radially from the axis of the shaft.

In other embodiments, when the deflectable member is in the first position, the shaft and the deflectable member are substantially disposed in a long, thin configuration slidable through several centimeters of a cannula inserted through a minimally-invasive surgical incision.

In still other embodiments, devices of the invention provide for the tip of the needle to comprise a sharpened point that pierces through tissue when the needle is advanced into tissue. Further, the needle comprises a hook that carries the first end of the suture. The first end of the suture is attached to a detachable loop that is pulled by the hook. When the needle carries the first end of the suture to the location on the deflectable member spaced away from the axis of the shaft, the deflectable member captures the detachable loop. When the needle retracts into the shaft, the deflectable member retains the detachable loop and detaches the detachable loop from the first end of the suture. In a further embodiment, when the device delivers the suture into tissue and is pulled away from the tissue, the deflectable member carries the detached detachable loop away from the tissue.

In embodiments, the deflectable member comprises a grid of catching ports spaced at regular intervals. The device may include a suture attachment rod or similar connector device to assist with the connection of the detachable loop to the insertion needle during surgery. For example, the product may be supplied as a kit that includes the device and one or more suture attachment rods. Optionally, each suture attachment rod may be pre-attached to one detachable loop.

A detachable loop or detachable member of the disclosure may be adapted for quick connection to a suture during surgery. For example, the detachable loop or detachable member may include a snap mechanism such as a coupling loop that provides for connection of the suture to the detachable member manually, e.g., while using the device. In an exemplary coupling loop, the suture may be pressed against a gap of the coupling loop such that the suture passes through the gap and is caught within the coupling loop.

In another aspect, the invention provides a suture delivery device comprising a body with a handle, an elongated shaft extending from the body, a suture within the shaft, a needle disposed within the shaft and slidable out of the shaft through the target tissue, and a catch portion of the shaft. The suture comprises a detachable member attached to a first end of the suture. The needle comprises a hook engaged with the detachable member to pull the suture into and through the tissue. When the needle slides through tissue, the hook emerges from the tissue and engages the detachable member with the catch. When the suture is within the tissue and the shaft is pulled away from the tissue, the catch pulls the detachable member off of the suture and away from the tissue, leaving only the suture within the tissue with ends extending from the tissue.

In embodiments, the detachable member of the device is attached to a terminus of the suture and comprises an open loop. In one embodiment, the suture is made of synthetic polymer fibers and the detachable member is made of stamped or molded or cast metal or plastic.

In another embodiment, the catch is provided by a deflectable member that can be deflected away from the axis of the shaft. The needle comprises a shape memory that is held in a straight configuration when the needle is confined within the shaft and, when the needle slides out of the shaft assumes a curved shape to bring the hook and detachable member to the catch on the deflectable member. Further, the deflectable member comprises a plurality of catch positions to receive the detachable member upon exit from the tissue. The deflectable member can be positioned along an axis of the shaft with the shaft and the deflectable member substantially disposed in a long, thin configuration slidable through several centimeters of a cannula inserted through a minimally-invasive surgical incision. When the device is pulled away from the tissue, the detachable member remains connected to the catch on the deflectable member and is removed from the suture, leaving only the suture in the tissue with ends extending from the tissue. Further, the catch catches the detachable member when the deflectable member is deflected away from an axis of the shaft and the needle is advanced out of the shaft to form the suture into a stitch in the tissue, wherein a first end of the stitch is made substantially along the axis of the shaft, and a second end of the stitch is made at a position displaced radially from the axis of the shaft.

In other embodiments, the delivery needle comprises a pointed tip with the hook just proximal to the pointed tip along the delivery needle.

In still another embodiment, the delivery needle is retracted back into the shaft after the detachable member is caught by the catch. In devices of the invention, the suture is delivered into the tissue and back out of the tissue at entry and exit points defining a stitch length wider than a diameter of the shaft.

In embodiments, the catch is positioned on a bendable appendage to the shaft and wherein the catch receives and catches the detachable member at a tissue exit point substantially spaced away from an axis of the shaft. Further, the length of the suture stich can be more than at least double the diameter of the shaft.

Alternatively or additionally, the device passes the suture through tissue, removes the detachable member from the suture, and leaves only the suture in the tissue with first and second ends of the suture extending out of the tissue at respective first and second exit locations spaced apart a length on a surface of the tissue.

### BRIEF DESCRIPTION OF THE DRAWINGS

FIG. 1 shows a suture delivery device of the invention.

FIG. 2 shows the device with the deflectable member pressed against tissue

FIG. 3 shows the deflectable member in a first position.

FIG. 4 illustrates the exit port of the shaft, the deflectable member, the receiving mechanism of the deflectable member, and the curved path of the needle as it is actuated to exit the shaft housing and travel to the deflectable member.

FIG. 5 further illustrates a side view of the distal end of the shaft with the needle leaving the exit point in a substantially curved shape and delivering the suture to the deflectable member.

FIG. 6 shows the suture in the tissue with an end extending from the entry point and an end extending from an exit point of the tissue.

FIG. 7 shows an isometric view of an embodiment of the device with a detachable member as a detachable loop attached to an end of the suture which has been carried by the needle through the tissue to a locking port on a deflectable member of the device.

FIG. 8 shows an embodiment of the needle with a hook at the tip to catch the suture coupled to a detachable loop, hook or crook.

FIG. 9 illustrates an embodiment of the device in which the suture with a detachable member is housed within the shaft.

FIG. 10 shows an embodiment of the suture with a detachable member shaped as a detachable loop and attached at an end of the suture.

FIG. 11 illustrates a detachable member shaped as a detachable loop received and locked by the deflectable member.

FIG. 12 shows an isometric view of the deflectable member in a second position disposed substantially deflected from the axis of the shaft with the tissue-facing surface pressed against the tissue and the needle carrying the suture with a detachable member to the receiving mechanism of the deflectable member.

FIG. 13 illustrates the shaft of the device with the deflectable member presenting a tissue facing surface with dotted arrow indicating general movement of the deflectable member in embodiments.

FIG. 14 shows the deflectable member in a second position disposed substantially deflected from the axis of the shaft with the tissue-facing surface pressed against tissue.

FIG. 15 shows an embodiment of the device with the tissue-facing surface of the deflectable member pressed against tissue at an angle substantially deflected from the angle of the axis of the shaft, and the needle with the suture entering the tissue at a first entry point and exiting the tissue at a second exit point to define a stitch width.

FIG. 16 illustrates an embodiment of the deflectable member coupled to the shaft.

FIG. 17 shows a member shaped as a detachable clip or tab.

FIG. 18 illustrates the detachable member as a detachable clip attached to the needle.

FIG. 19 shows the needle.

FIG. 20 shows the needle being captured.

FIG. 21 shows a deflectable member pulling a detachable member away.

FIG. 22 illustrates the detachable member attaching to an embodiment of the needle with a notched slot or groove to connect to the detachable member.

FIG. 23 shows a detachable member shaped as a y-shaped member.

FIG. 24 shows embodiments of the curved needle with a notch, groove, or cut-out for connecting to detachable member embodiments.

FIG. 25 illustrates an embodiment of the detachable member shaped as a winged connector with a coupling loop for connecting to the suture.

FIG. 26 illustrates of an embodiment of the suture threaded through the coupling loop of the detachable member shaped as a winged connector.

FIG. 27 illustrates an embodiment of the suture with a loop at a terminus connected to the detachable member via the coupling loop of the detachable member.

FIG. 28 shows an embodiment of a connector device for connecting the detachable member to the needle.

FIG. 29 illustrates an embodiment of the connector device shaped to attach to an embodiment of the detachable member.

FIG. 30 illustrates an embodiment of the connector device connected to an embodiment of the detachable member.

FIG. 31 illustrates an embodiment of the connector device, with the detachable member connected, for connecting to the needle housed within the shaft.

FIG. 32 illustrates an embodiment of the connector device inserting the detachable member into the shaft to connect to the needle therein.

FIG. 33 illustrates an embodiment of the connector device connecting the detachable member to the needle housed within the shaft.

FIG. 34 illustrates an embodiment of the detachable member connected to the needle housed within the shaft with the connector device removed.

FIG. 35 shows a cross-section view of an embodiment of the shaft with the needle housed within the shaft and the detachable member attached to both the needle and the suture.

FIG. 36 illustrates an embodiment of the needle, attached to the detachable member with the suture attached, exiting the shaft.

FIG. 37 illustrates an embodiment of the device with a detachable member is a clip embodiment attached to an end of the suture which has been carried by the needle through the tissue to a locking port on a deflectable member of the device.

### DETAILED DESCRIPTION

The invention provides a device for delivering surgical sutures. Significantly, the device is capable of delivering sutures of any size stitch-width. For minimally invasive surgeries such as laparoscopic surgery or endoscopic surgery, the device allows for stitch widths that are not constrained by the diameter of the shaft of the instrument. The device may also be used to suture any type of anatomical tissue for any type of anatomical surgery such as organ or tissue repair. The device may be used for open surgery or minimally invasive surgeries, including laparoscopic or endoscopic surgeries, and may be used with catheters and other small or large diameter tubular or hollow, cylindrical members providing access to small cavities such as veins or arteries, as well as large cavities such as the abdomen. The device is capable of adapting to and adjusting to different tissue surfaces and incision sites for a variety of stitch widths or lengths to meet surgical suturing needs.

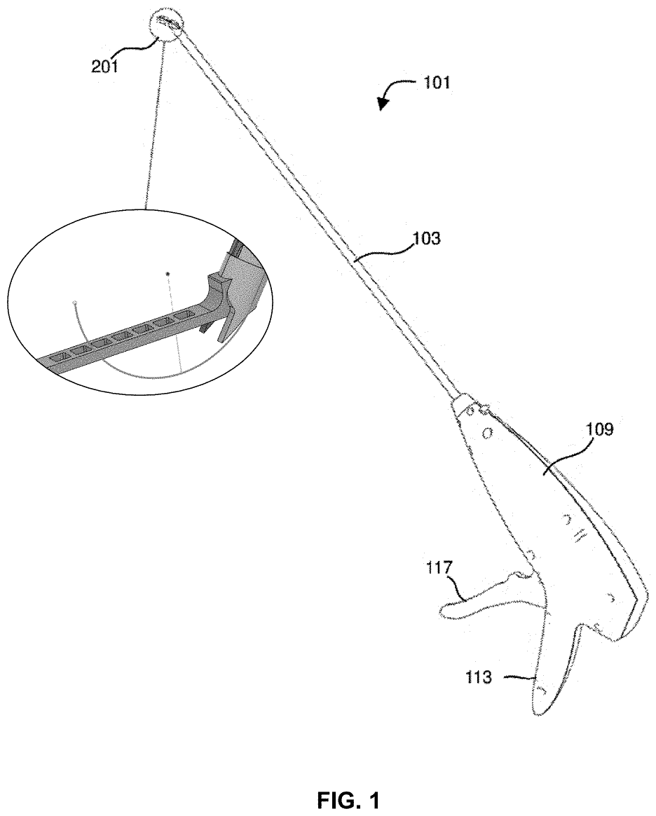

FIG. 1 shows a suturing device 101 according to embodiments of the disclosure. The suturing device 101 may include an elongated shaft 103 at the proximal end of a body 109. The device may have a handle 113 attached to the body. The body 109 may be coupled to a trigger 117 operably engaged with a driver member and moveable relative to the handle 113. Importantly, and as described in more detail below, the distal end of the elongated shaft terminates in a deflectable member 201 coupled to the distal end of the elongated shaft. The deflectable member presents a tissue-facing surface at a position in line with the axis of the shaft or substantially deflected therefrom. The flexibility of placing the tissue-facing surface in line with or at an angle deflected from the axis of the shaft allows a needle to penetrate tissue at a first entry point and exit the tissue at a second exit point such that the stitch width may be wider than the diameter of the shaft and is thus not limited to the diameter of a laparoscopic or other instrument.

The body 109 may be fixedly coupled to the shaft 103 at the proximal end. The body may include an actuator mechanism for moving and controlling a driver member housed within the body 109 and extending through the shaft 103. For example, the body 109 may be coupled to a trigger 117 operably engaged with the driver member and moveable relative to the handle 113. The actuator mechanism may transfer the motion of squeezing the trigger from the hand through a gear train to the distal end of the shaft where a pin-slot mechanism may actuate the deflectable member 201. The actuator mechanism may use mechanisms or features as described in U.S. Pat. Nos. 6,053,908 and 5,344,061, and in Miller, 2008, Mechanical design for modularity of laparoscopic tools, ASME J Med Dev 2(2):027508, all incorporated by reference.

The delivery tip 111, e.g., at a distal segment 112 of the shaft, may be articulated, allowing the surgeon to orient the delivery tip 111 suitable for an optimal approach to the target. For example, the shaft may have a living hinge or joint 121 some distance proximal to the terminus (e.g., a few centimeters). The joint 121 can be controllable from the handle by, for example, rotating a controller element 131, which may be a knob, thumbwheel or lever on the handle to pull via a cable through the shaft, which pulls the distal delivery tip 111 to an off-axis delivery position. This articulation may be in addition to the deflectable member 201. In fact, the combination of the hinge or joint 121 plus the deflectable member creates a form of double jointed shaft in which the distal segment 112 can bend away from the proximal and then the deflectable member and deflect away from an axis of the distal segment.

As with the distal segment 112, the deflectable member 201 may be controllable from the handle by, for example, rotating a tip controller element 132, which may be a knob, thumbwheel, or lever on the handle to pull via a cable through the shaft to cause the deflectable member to deflect into a desired angle with the shaft axis. In any of the embodiments, the deflectable member 201 angle could be actively controlled (by rotating the a tip controller element 132 such as a lever at the handle) in order to control the penetration depth of the needle and suture.

In preferred embodiments, the design of the trigger, controller, and actuator allows the user to control the angle of the shelf using a knob or lever on the handle. In addition to better device insertion and deployment of the deflectable member, the design allows the surgeon to control the penetration depth by controlling the angle of the deflectable member. The trigger may be a mechanism coupled to the body that the operator may push, pull, dial, or slide such that the movement actuates the deflectable member to assume any range of positions, for example, bend, squeeze, pull, or push such that the deflectable member may fold flat or bend at any angle.

In embodiments the trigger 117 may be coupled to the body 109 and may extend from the handle 113, such that the trigger is operably engaged with the driver member and moveable relative to the handle 113. Any suitable engagement mechanism may be used. For example, the trigger may present a curved, geared, surface within the body 109. The curved geared surface may engage a geared slot wheel through any optional stepper gears that increase or decrease a magnitude of rotation imparted when the trigger 117 is squeezed to rotate about a pivot. The geared slot wheel within the handle 109 may include an eccentric slot and a proximal end of the driver member may have a pin engaged into the slot, wherein squeezing the trigger causes the slot wheel to rotate. The eccentric slot pushes the pin in a direction that includes displacement parallel to an axis of the shaft 103. The displacement pushes the driver member in a direction distal along the shaft 103 and then pulls the driver member back in a proximal direction. Thus, squeezing the trigger causes the driver member to translate along the shaft outward and back to engage a needle operably coupled to the distal end of the driver member. The relation of the needle and deflectable member is discussed in detail below. Any suitable geared trigger with slot wheel and pin that may be modified for use with the invention may be used, and as is shown in U.S. Pat. No. 8,535,339, incorporated by reference.

The handle 113 may be ergonomically designed for optimal usability, comfort, and haptic feedback of the device, for example, as described in Alleblas, 2016, Ergonomics of laparoscopic graspers and the importance of haptic feedback: the surgeons' perspective, Gynecological surgery 13(4), 379-384, incorporated by reference. In embodiments, the handle 113 may be configured for use with and to couple to a robotic system. The robotic system may have one or more computers, wrist-like articulation at the body and at the distal end of the shaft, and motors for actuating the suture delivery device to enable interaction with the body 109, handle 113, and the deflectable member 201 at the distal end of the shaft. The handle 113 may include a handle control, thumb control, or mixed control or any such features as described in Anderson, 2016, Robot-like dexterity without computers and motors: a review of hand-held laparoscopic instruments with wrist-like tip articulation, Expert Rev Med Dev, 13(7):661-672, incorporated by reference. The handle may be a grip that can be squeezed, and which is operably coupled to the driver member. Alternatively, the handle 113 may be operably coupled to a robotic function.

The elongated shaft 103 extending from the body may be dimensioned for minimally invasive surgery. Minimally invasive procedures, for example laparoscopic surgery, encompass surgical techniques that limit the size of the incision needed. Trocars are placed through the abdomen during laparoscopic surgery. A trocar is a medical device made up of an awl, a cannula or hollow tub, and a seal. Therefore, in some embodiments, the diameter of the shaft 103 of the device, may be generally smaller than a standard trocar. The shaft is preferably long enough to reach a surgical target, e.g., at least about 15 cm long, preferably at least 25 cm long. As discussed below, the shaft terminates in an exit port and is coupled to the deflectable member 201 at the distal end of the shaft. The deflectable member 201 is coupled to the distal end of the shaft 103.

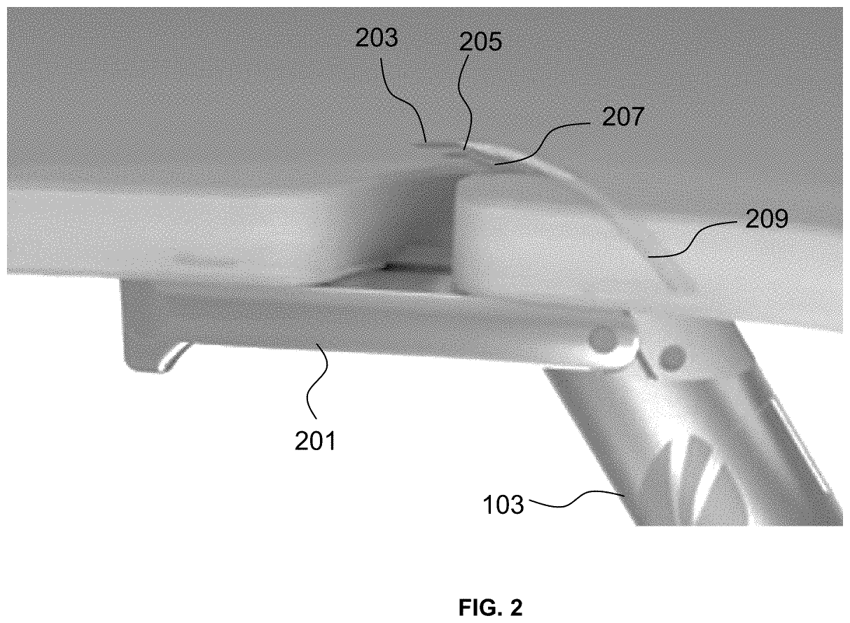

FIG. 2 illustrates a side view of an embodiment of the shaft 103 with the deflectable member 201 protruding from the distal end of the shaft. As is discussed in more detail below, in embodiments, the device may include a needle 203 comprising a hook 205, or a hook-shaped mouth to catch or grab a detachable loop 207 attached to the suture 209.

In the illustration, the deflectable member 201 is in a second position. Embodiments of the deflectable member may include a shelf or foot that folds radially away from the extended shaft once the device is inserted, to allow for delivery of stitches to locations that are off-axis from the delivery shaft. In other embodiments, the deflectable member may be housed within the shaft and deployed to the surface of tissue by operation of a driver member operably coupled to the deflectable member and actuated by a trigger coupled to the body. The deflectable member may be flexibly coupled to the shaft such that the deflectable member is moveable between at least a first position and a second position. In embodiments, the design of the trigger, controller, and actuator allows the user to control the angle of the shelf using a knob or lever on the handle. In addition to better device insertion and deployment of the deflectable member, the design allows the surgeon to control the penetration depth by controlling the angle of the deflectable member. The trigger may be a mechanism coupled to the body that the operator may push, pull, dial, or slide such that the movement actuates the deflectable member to assume any range of positions or angles. For example, operation of the trigger may actuate the deflectable member to bend, squeeze, pull, or push such that the deflectable member may fold flat or bend at any angle.

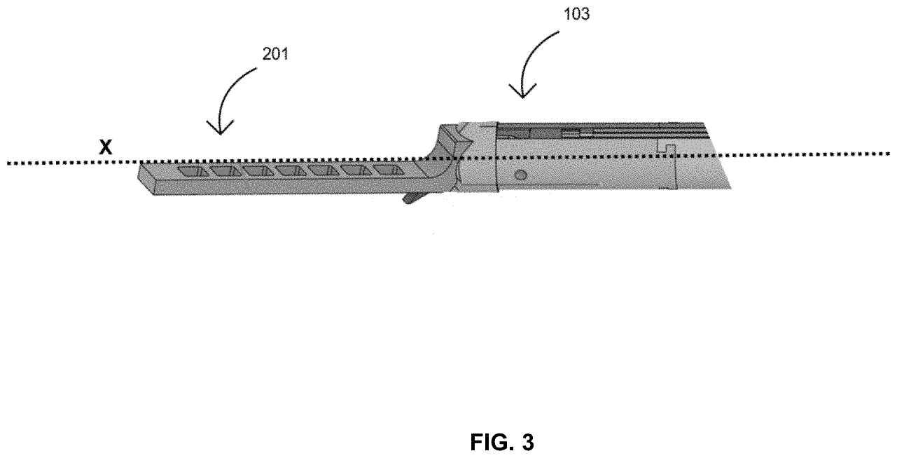

FIG. 3 shows the deflectable member 201 in a first position extending along an axis of the shaft 103. The axis of the shaft is depicted by dotted line X and may be thought of as a reference line to measure the angle of deflection by the deflectable member in relation to the fixed position of the shaft.

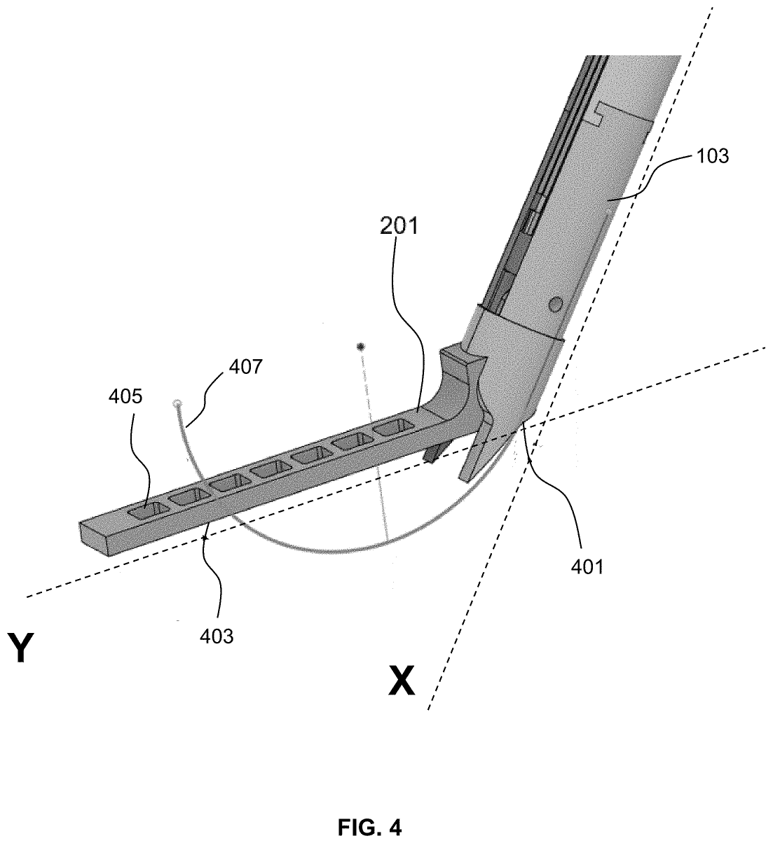

FIG. 4 Illustrates the deflectable member 201 in a second position extending from the distal end of the shaft 103 at an angle offset from the axis X of the shaft. The deflectable member 201 may be coupled to the shaft 103 by a hinge or pivot that allows the deflectable member 201 to rotate relative to the fixed axis of the shaft 103. The hinge may be any suitable hinge that allows for deflection from at least a first position to a second position relative to the fixed axis of the shaft, for example, a flexibly deformable material, a notch, a curved wrist-joint, hinge pin or other joint. In embodiments, the distal end of the shaft includes an exit port 401 from which the needle coupled with the suture exits.

Preferably, the deflectable member presents a tissue-facing surface 403, which, when the deflectable member is in the second position, the tissue-facing surface 403 extends along the line Y. The second position of the deflectable member may be substantially offset from the axis of the shaft such that line Y and the axis X of the shaft form an acute angle. However, the deflectable member may be deflected away from the axis of the shaft at any angle from 0 degrees to 180 degrees. The deflectable member 201 may occupy the first position (extending along the axis X) or second position (deflected from the axis X). The deflectable member may be biased to preferentially occupy either the first position or the second position, with the ability to be elastically deformed into the other of the first and second positions.

In embodiments, the distal end of the shaft includes one or more pointed tips adapted to enable the surgeon to facilitate tissue approximation prior to suture deployment.

In embodiments, the deflectable member may include a receiving mechanism 405 to receive suture the needle. As described in more detail below, the curved needle travels along a curved path 407 from the exit port 401 of the shaft 103 to the receiving mechanism 405 on the deflectable member 201. The receiving mechanism 405 on the deflectable member 201 may be in spatial communication with an exit port 401 of the shaft 103. In some embodiments, the receiving mechanism 405 of the deflectable member is characterized by having a narrow width along the tissue-facing surface 403 in order to hold the receiving end of the suture. As described in detail below, in embodiments, the receiving mechanism may consist of a series of grids or holes that function as locking ports to receive a detachable member attached to suture.

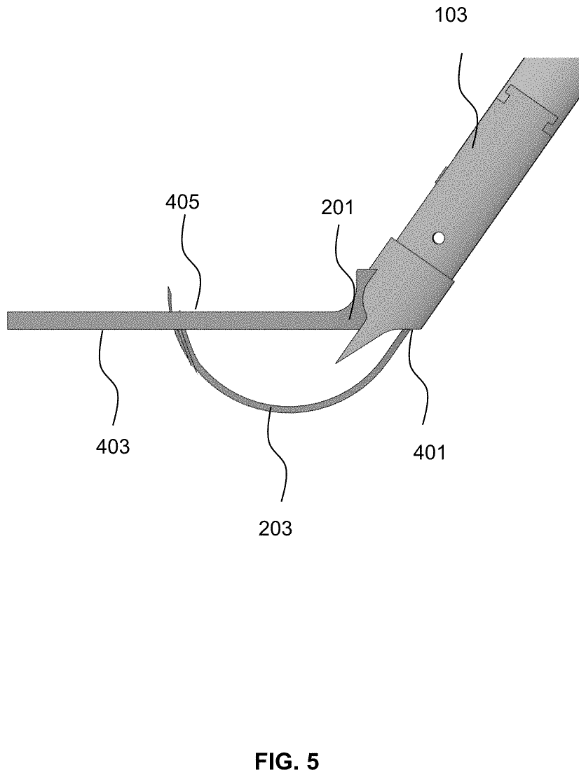

FIG. 5 shows the deflectable member 201 coupled to the shaft 103 and illustrates the exit port 401 in which the needle 203 has exited along a curved path to the receiving mechanism 405 of the deflectable member. Illustrated is an embodiment in which the deflectable member 201 is biased to naturally occupy the second position, deflected away from the shaft axis. However, in this embodiment, while the shaft 103 is delivered through a cannula, the bore of the cannula constrains the deflectable member 201 into the first position (e.g., bends it into the straight extension from the shaft axis). Another default may be used in which the deflectable member is biased to naturally occupy the first position, extending along the shaft axis, but the tissue-facing surface 403 may be pressed against tissue to elastically deform the deflectable member into the second position. In yet other embodiments, the deflectable member is neutral (e.g., it has a hinge with modest but effective friction to hold the deflectable member in whatever position into which it is moved).

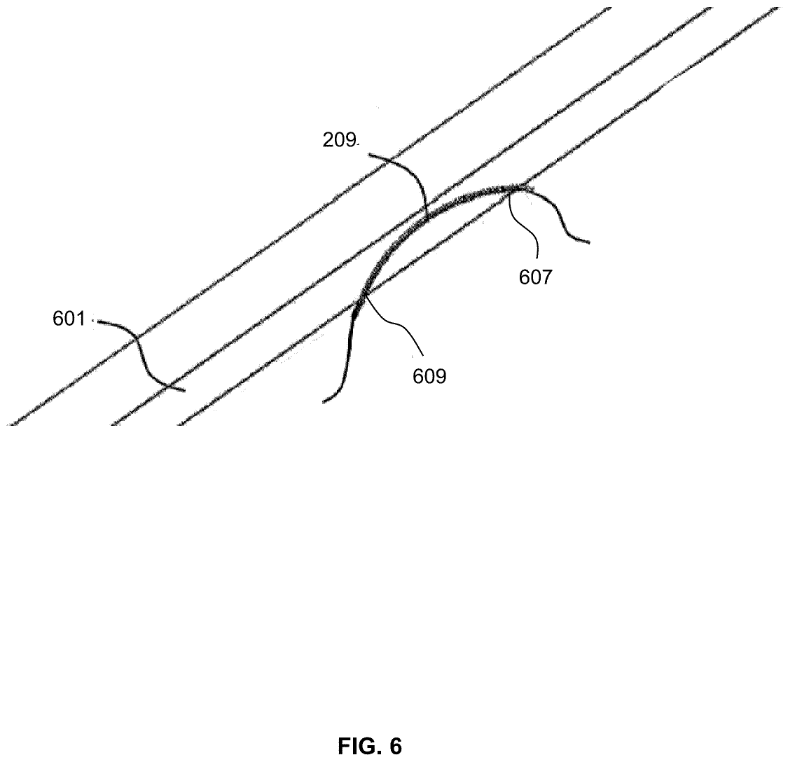

FIG. 6 illustrates delivery of the suture 209 into the tissue 601. Devices of the invention provide for delivering surgical sutures. In embodiments, the device delivers the suture into the tissue at a first entry point 607 and back out of the tissue at a second exit point 609 leaving the suture extending within the tissue with a first end emerging from the tissue at the exit point and the second end emerging from the tissue at the entry point. Thus, the entry point 607 and the exit point 609 define a stitch length. In embodiments, the stitch length is substantially larger than the diameter of the shaft 103. In other embodiments, the needle passes the suture through tissue and leaves the suture in the tissue with first and second ends of the suture extending out of the tissue as described above. The first and second exit locations are spaced apart a length on the surface of the tissue.

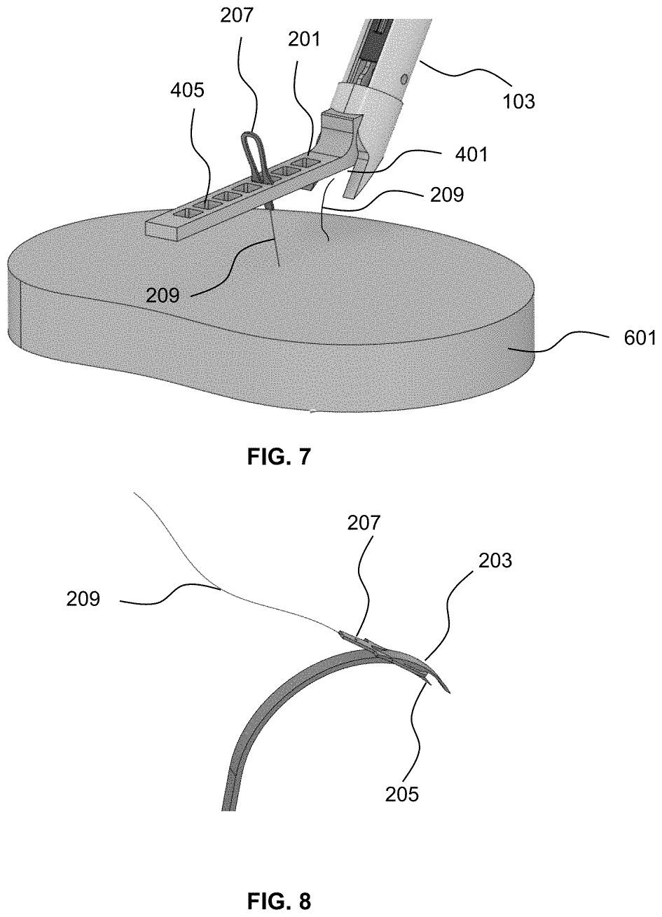

FIG. 7 illustrates an embodiment of the deflectable member 201 coupled to the distal end of the shaft 103 and in a second position off-set from the axis of the shaft. The device may have an exit port 401 at the distal end of the shaft from which the needle carries the suture 209 through the tissue 601 to the receiving mechanism 405 on the deflectable member 201. The suture may be coupled to a detachable member such as a detachable loop 207 which locks into the receiving mechanism 405 of the deflectable member 201. In embodiments, the device may include a needle delivery slot housed within the distal end of the shaft and opening to the exit port 401. The needle may be held within the distal end of the shaft 103 at the exit port 401 and may be operably coupled to the distal end of a driver member disposed within the shaft. The driver member may be actuated to push the needle with the suture 209 out of the shaft and into the tissue 601. Thus, the needle delivery slot allows the driver member to push the needle through the exit port 401 and into the surface of the tissue at a first entry point and out of the tissue at a second exit port. The exit port 401 may therefore define a space through which the driver member may push the needle out of the shaft 103, and may be in spatial communication with a receiving mechanism located on the deflectable member. In some embodiments, the devices of the invention include a spring-loaded mechanism in the handle 113 in order to facilitate faster application which may be necessary for better penetration through tissue.

In embodiments, the receiving mechanism 405 receives the suture 209 after the needle exits the tissue and delivers the suture to the receiving mechanism on the deflectable member 201. The receiving mechanism may be any mechanism capable of capturing and holding the end of the suture once delivered by the needle. For example, the receiving mechanism of the deflectable member may be an anchor, hook, clamp, grid, snap, clip, locking holes, or jaw mechanism. Alternatively, the receiving mechanism may be an adhesive to which suture will adhere. In some embodiments, the receiving mechanism is a series of locking ports with square holes on the deflectable member or shelf.

Devices of the invention provide for delivering surgical sutures. In embodiments, the tip of the needle carries a first end of the suture to the location of the receiving mechanism on the deflectable member spaced away from the axis of the shaft. The suture may be housed within the shaft, for example as a spool or as predetermined lengths of suture. The suture material may be any material as is known to one skilled in the art such as absorbable polyglycolic acid, polylactic acid, Monocryl and polydioxanone, as well as non-absorbable materials such as nylon, polyester, PVDF, or polypropylene. The suture may be a biodegradable polymer such as poly(lactic acid) (PLA), a polymer of glycolic acid (PGA), polycaprolactone (PCL), either braided or not braided. The suture may be a non-absorbable polymer such as Nylon or polypropylene, either braided or not braided. Alternatively, the suture may be a biologic (non-synthetic) material such as collagen or silk. In embodiments, the suture may be a metal, such as stainless steel, either braided or not braided. The suture may be any thread, braid, or monofilament that may be tied by knotting. The suture may be housed in a spool and may be capable of being cut into any arbitrary length for tying and knotting. The suture may have a detachable member such as a detachable loop 207 on the distal end that is caught by the needle to carry the suture through the tissue and deliver the suture to the receiving mechanism of the deflectable member. The detachable loop 207 serves as a suture connector device, connecting the suture to the needle of the device. The detachable loop may be of a shape designed to connect to the curved needle, connect to the suture, and to lock on the receiving mechanism of the deflectable member. For example, in embodiments, the detachable loop may be flexibly deformable to fit within openings on the receiving mechanism but may also include wings, flanges, or ears capable of expanding after entering the locking port to prevent the detachable loop from retracting back through the opening. In embodiments, the ears or flanges allow the detachable loop to passively lock onto the deflectable member. For example, the detachable loop may be adapted to pass through a groove at the shelf and then be caught by the receiving mechanism of the deflectable member, for example by having two lateral barbs at the side of the loop.

The receiving mechanism of the deflectable member may catch the detachable loop 207 to hold the end of suture while the needle is retracted back into the shaft. In embodiments described in more detail below, the detachable member may be a loop or crook that is caught by the needle. In other embodiments the suture may be coupled to a detachable member which may be a loop, hook, clip, clasp, shackle, tab, or y-shaped member. In embodiments, the detachable loop 207 is locked into the receiving mechanism 405 of the deflectable member 201 for removal from the surgical site. In embodiments, the receiving mechanism is a series of locking ports with square holes on the deflectable member. The detachable member will peel off from the needle after passing inside one of the square holes on the deflectable member. The peeled detachable member will thereafter not be able to move back through the square holes due to the geometric constraints of the design of the detachable member.

Additional embodiments of the invention may be related to an insertion device for inserting suture into a guide slot for feeding to the needle end. In embodiments, devices of the invention provide for the needle to catch the end of the suture and carry the suture along a curved path into the tissue at a first entry point and out of the tissue at a second exit point to deliver the suture to the receiving mechanism on the deflectable member.

The movement of the needle along the path may be made possible by the action of the driver member. The driver member may retract the needle back into the shaft once the suture has been received by the receiving mechanism of the deflectable member. Thus, in some embodiments, the operation of the trigger causes the driver member to push the needle with an end of suture attached to the needle, out of the exit port and into the tissue at a first entry point along a path in alignment with the axis of the deflectable member. The needle then exits the tissue at a second exit point to deliver the suture to the receiving mechanism of the deflectable member. Once the deflectable member has received the suture, the needle is retracted back into the shaft by operation of the driver member leaving the ends of the suture extending from the tissue surface at the entry and exit points.

In further embodiments, the receiving mechanism of the deflectable member receives the end of suture as delivered from the needle and holds the end of the suture when the needle is retracted back into the shaft. The needle may be retracted back through the exit port of the shaft and into the shaft by the driver member. In embodiments, the deflectable member comprises a grid of catching ports spaced at regular intervals. The grid of catching ports may be used as the receiving mechanism for the suture. The grid of catching ports may be the same size or have varying openings and spacings.

FIG. 8 illustrates one embodiment of a needle 203 of the device, which includes a hooked mouth 205 for catching a detachable loop 207 attached to the suture 209.

The needle is preferably made of a super-elastic material such that when the needle is housed within the shaft, the needle is in a substantially straight configuration and when the needle is advanced out of the shaft the needle assumes a curved shape. For example, in embodiments, once the needle is retracted to the shaft, it is held straight but is not parentally deformed. The material may be, for example, an alloy such as copper-aluminum-nickel or nickel-titanium alloy, or a super-elastic polymer. When the needle is advanced out of the shaft with the deflectable member in a position deflected from the axis of the shaft, the needle is in a curved shape that carries the tip of the needle to a location on the deflectable member spaced away from the axis of the shaft. In embodiments, the needle may have a sharpened point that pierces through the tissue when the needle is advanced into the tissue. The needle may comprise a hook that catches the first end of the suture and carries it to deliver the suture at the receiving mechanism of the deflectable member. In other embodiments, the needle may have a groove, notch, fitted slot, slotted hole, or cutout to catch the first end of the suture.

In embodiments, the invention provides for suture to be attached to a detachable member. The detachable member may be a loop or crook that is caught by the needle. In other embodiments the suture may be coupled to a detachable member which may be a loop, hook, clip, clasp, shackle, tab or y-shaped detachable member. In embodiments, the suture is connected to the detachable member at the medical facility by medical staff. In other embodiments, the suture is pre-connected to the detachable member during a manufacturing process.

In one embodiment, the detachable member may be a detachable loop 207 that is attached to the suture 209 on the distal end. The detachable loop functions as a connector to connect the suture and the needle 203. The first end of the suture is attached to a detachable loop 207 that can be caught by the needle hook 205 and pulled by the hook through the tissue to deliver both the suture and the detachable loop to the receiving mechanism on the deflectable member. Therefore, when the needle carries the first end of the suture with the detachable loop to the receiving mechanism on the deflectable member, with the deflectable member spaced away from the axis of the shaft, the receiving mechanism of the deflectable member captures the detachable loop. In other embodiments, the detachable member may be connected to the needle by the physician before insertion into the shaft. Alternatively, the detachable member may be placed by the physician in a collection area within the shaft where it is connected to the needle as the needle is deployed out of the device. In still other embodiments, the detachable member may be pre-loaded into a cartridge device that fits within the shaft and automatically connected to the needle by a dedicated magazine mechanism.

The detachable loop may be shaped to fit into the receiving mechanism of the deflectable member such that when it is caught it will not retract back out when the needle is withdrawn. For example, the receiving mechanism of the deflectable member may include locking ports or grid which catch the detachable loop and hold the end of suture while the needle is retracted back into the shaft. In embodiments, the detachable loop may be rigid or flexibly deformable such that the shape operates to fit the detachable loop within a grid of locking ports on the deflectable member. The detachable loop may be flexibly deformable to fit within openings on the receiving mechanism but may also include wings, flanges, or ears capable of expanding after entering the locking port to prevent the detachable loop from retracting back through the opening. In embodiments, the ears or flanges allow the detachable loop to passively lock onto the deflectable member.

The needle carries the detachable loop and suture through a locking port of the deflectable member such that the shape of the detachable loop locks the detachable loop into the receiving mechanism of the deflectable member and prevents the detachable loop from retracting back through the locking port as the needle is retracted. The receiving mechanism of the deflectable member may catch the detachable loop to hold the end of suture while the needle is retracted back into the shaft. Thereafter, when the needle retracts back into the shaft, the receiving mechanism of the deflectable member retains the detachable loop and detaches the detachable loop from the first end of the suture.

In embodiments, when the needle is advanced out of the shaft to form the suture into a stitch in the tissue, the first end of the stitch at the first entry point may be made substantially along the axis of the shaft, and the second end of the stitch, the end of which is received by the deflectable member, may be made substantially displaced radially from the axis of the shaft. Therefore, because of the possible positions of the deflectable member, the length and direction of the stitch is not constrained by the position of the shaft.

Further, when the deflectable member is in the first position, the first position is along the axis of the shaft such that the deflectable member and the shaft are substantially disposed in a long, thin, configuration slidable through several centimeters of a cannula inserted through a minimally-invasive surgical incision.

The invention provides for a suture delivery device comprising a body having a handle, an elongated shaft extending from the body, a suture housed within the shaft, wherein the suture comprises a detachable member shaped as a detachable loop attached to a first end of the suture, a needle disposed within the shaft and slidable out of the shaft through target tissue, the needle comprising a hook engaged with the detachable member to pull the suture into and through the tissue, and a catch on a portion of the shaft wherein when the needle slides through the tissue, the needle comprising the hook engaged with the detachable member to pull the suture into and through the tissue.

Devices of the invention generally include a body attached to an elongated shaft at the proximal end of the shaft, a handle attached to the body. In embodiments, the device may have a deflectable member coupled to the distal end of the elongated shaft. In embodiments, the device passes the suture through tissue, removes the detachable member from the suture, and leaves only the suture in the tissue with first and second ends of the suture extending out of the tissue at respective first and second exit locations spaced apart a length on a surface of the tissue.

The body may be fixedly coupled to the shaft at the proximal end. In embodiments, the coupling may be a rotational coupling with or without the ability to lock the body into a fixed coupling with the shaft. The body may include an actuator mechanism for moving and controlling a driver member housed within the body and extending through the shaft. For example, the body may be coupled to a trigger operably engaged with the driver member and moveable relative to the handle. The actuator mechanism may transfer the motion of squeezing the trigger from the hand through a gear train to the distal end of the shaft where a pin-slot mechanism may actuate the needle. Alternatively, the actuator mechanism may comprise a triggering mechanism that is known in the art such as, for example, in U.S. Pat. Nos. 6,053,908 and 5,344,061, and as reported in Miller, D. J., 2008, Mechanical Design for Modularity of Laparoscopic Tools, ASME. J. Med. Devices 2(2): 027508, each of which are incorporated by reference in their entirety herein. In embodiments, the design of the trigger, controller, and actuator allows the user to control the angle of the shelf using a knob or lever on the handle. In addition to better device insertion and deployment of the deflectable member, the design allows the surgeon to control the penetration depth by controlling the angle of the deflectable member. The trigger may be a mechanism coupled to the body that the operator may push, pull, dial, or slide such that the movement actuates the deflectable member to assume any range of positions, for example, bend, squeeze, pull, or push such that the deflectable member may fold flat or bend at any angle.

In embodiments the trigger may be coupled to the body and may extend from the handle, such that the trigger is operably engaged with the driver member and moveable relative to the handle. Any suitable engagement mechanism may be used. For example, the trigger may present a curved, geared, surface within the body. The curved geared surface may engage a geared slot wheel through any optional stepper gears that increase or decrease a magnitude of rotation imparted when the trigger is squeezed to rotate about a pivot. The geared slot wheel within the handle may include an eccentric slot and a proximal end of the driver member may have a pin engaged into the slot, wherein squeezing the trigger causes the slot wheel to rotate. The eccentric slot pushes the pin in a direction that includes displacement parallel to an axis of the shaft. The displacement pushes the driver member in a direction distal along the shaft and then pulls the driver member back in a proximal direction. Thus, squeezing the trigger causes the driver member to translate along the shaft outwards and back to engage the needle operably coupled to the distal end of the driver member to catch the detachable member of the suture. Any suitable geared trigger with slot wheel and pin that may be modified for use with the invention may be used, and as is shown in U.S. Pat. No. 8,535,339, incorporated by reference.

The handle may be ergonomically designed for optimal usability, comfort, and haptic feedback. In embodiments, the shaft may be configured for use with a robotic system. The robotic system may have a one or more computers, mechanical articulation at the body and/or at the distal end of the shaft, and motors for actuating the suture delivery device to enable interaction with the body and handle and the needle at the distal end of the shaft. The handle may include a handle control, thumb control, or mixed control as is known to a person skilled in the art and described in Anderson, 2016, Robot-like dexterity without computers and motors: a review of hand-held laparoscopic instruments with wrist-like tip articulation, Expert Rev Med Dev 13(7):661-672, incorporated by reference. The handle may be a grip that can be squeezed, and which is operably coupled to the driver member. Alternatively, the shaft may be operably coupled to a robotic function.

The elongated shaft extending from the body may be dimensioned for minimally invasive surgery. Minimally invasive procedures, for example laparoscopic surgery, encompass surgical techniques that limit the size of incision needed. Trocars are placed through the abdomen during laparoscopic surgery. A trocar is a medical device made up of an awl, a cannula or hollow tub, and a seal. Therefore, in some embodiments, the diameter of the shaft 103 of the device, may be generally smaller than a standard trocar. The shaft is preferably long enough to reach a surgical target, e.g., at least about 15 cm long, preferably at least 25 cm long.

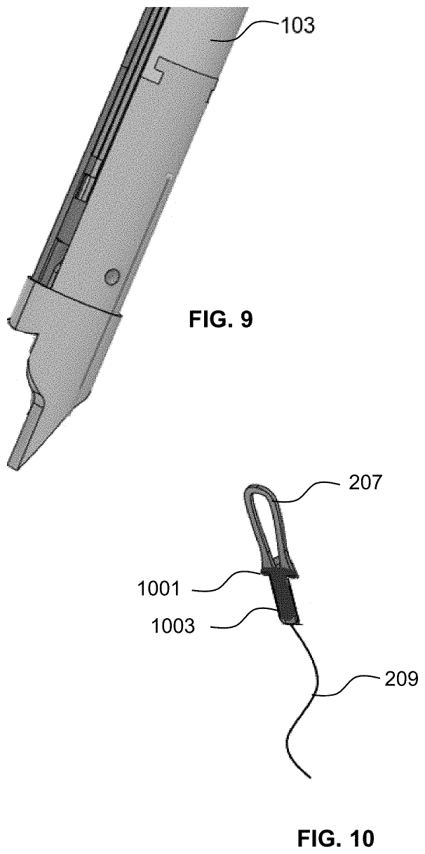

FIG. 9 illustrates the shaft 103 in which the suture and needle are housed.

FIG. 10 illustrates an embodiment of a detachable member shaped as a detachable loop 207. The detachable loop 207 functions as a suture connector to connect the suture 209 and the needle. The detachable loop may have ears 1001, flanges, or wings to allow for passive locking on the deflectable member. The detachable loop may be connected to the suture, for example, using a groove 1003, hole, slot, adhesive, or clip. The detachable loop may be shaped to fit into the receiving mechanism of the deflectable member such that when it is caught it will not retract back out when the needle is withdrawn. For example, the receiving mechanism of the deflectable member may include locking ports or a grid which catch the detachable loop and hold the end of suture while the needle is retracted back into the shaft. In embodiments, the needle comprises a hook-shaped mouth, such as a shark mouth shape, such that the needle can grab or hook the detachable loop and carry the detachable loop and the suture through the tissue to the receiving mechanism of the deflectable member.

The detachable member may be rigid or flexibly deformable such that the shape operates to fit the detachable member within a grid of locking ports on the deflectable member. In embodiments, the detachable loop may be flexibly deformable to fit within openings on the receiving mechanism but may also include wings, flanges, or ears capable of expanding after entering the locking port to prevent the detachable loop from retracting back through the opening. In embodiments, the ears or flanges allow the detachable loop to passively lock onto the deflectable member. In other embodiments described below, the detachable member may be, for example, a loop, hook, crook, tab, catch, clip, clasp, shackle, or y-shaped member. The detachable member may be made of a rigid or flexibly deformable material capable of passing through tissue at a first entry point, passing through the tissue, and exiting the tissue at a second exit point.

In embodiments, the suture with the detachable member attached to an end of the suture is pre-loaded into the device and housed within a guide slot of the shaft. The shaft may include a delivery slot extending to the exit port and also extending to a release port. In embodiments, each length of suture with a detachable loop attached is held within the shaft in a configuration such that the needle can hook the detachable loop upon exit from the shaft. In other embodiments, each length of suture with a detachable member attached is held within the shaft in a configuration such that the needle can attach to the detachable member upon exit from the shaft. In still other embodiments, the detachable member is attached to the needle before loading into the device.

In other embodiments, each length of suture with the detachable member is held within the shaft in a configuration such that the needle can catch the detachable member upon exit from the shaft. In still other embodiments, the device includes a mechanism for attaching the detachable member to the suture within the shaft as the suture is fed into the shaft.

Any number of lengths of suture with detachable members attached may be pre-loaded into the shaft housing. Subsequently, each operation of the trigger may catch the detachable member by the needle, deliver the suture into tissue at a first entry point and out of tissue at a second exit point, retract the needle, and advance another detachable member attached to the suture for catching by the needle. In other embodiments, the detachable member is attached to the end of the suture by the action of the needle and driver member. In still other embodiments, the suture is connected to the detachable member outside the housing and pre-loaded in the shaft housing. In other embodiments, the suture and detachable member are coupled within the housing.

In other embodiments, the device provides a needle operably coupled to a driver member housed within the shaft. In embodiments, the needle is housed within the distal end of the shaft and coupled to the driver member in a needle delivery slot at the exit port of the shaft. The needle is preferably made of a super-elastic material such that when the needle is housed within the shaft, the needle is in a substantially straight configuration and when the needle is advanced out of the shaft the needle assumes a curved shape. For example, in embodiments, once the needle is retracted to the shaft, it is held straight but is not parentally deformed. The material may be, for example, an alloy such as copper-aluminum-nickel or nickel-titanium, or a super-elastic polymer. In embodiments, when the needle is advanced out of the shaft, the needle is in a substantially curved shape. The needle may have a sharpened point that pierces through tissue when the needle is advanced into the tissue. The needle may comprise a hook that catches the detachable member on the end of the suture and carries it to deliver the suture into tissue at a first entry point and back out of the tissue at a second exit port. In embodiments the needle comprises a notch, clip, groove, fitted slot, slotted hole, catch, or cutout to catch the detachable member attached to the first end of the suture.

In embodiments, the distal end of the shaft includes an exit port. The needle is held within the distal end of the shaft at the exit port. The device may contain a needle delivery slot housed within the shaft and opening to the exit port. In embodiments, the needle may be operably coupled to the distal end of a driver member disposed within the shaft, in which the driver member is channeled and guided by a delivery slot that extends within the shaft. The exit port defines a space through which the driver member may actuate the needle to catch the detachable member of the suture and push the needle out of the shaft. Thus, the needle delivery slot allows the driver member to push the needle through the exit port, catch the detachable member of the suture, and push the needle holding the detachable member and suture into the surface of the tissue at a first entry point and out of the tissue at a second exit port. In some embodiments, the devices of the invention include a spring-loaded mechanism in the handle in order to facilitate faster application which may be necessary for better penetration through tissue.

Devices of the invention provide for delivering surgical sutures. In embodiments, the tip of the needle carries a detachable member attached to a first end of the suture into and out of tissue spaced away from the axis of the shaft. In embodiments, the suture may be housed within the shaft, for example as a spool or as predetermined lengths of suturing. The suture may be pre-loaded with a detachable member already attached to an end of the suture, or the driver member may be actuated to attach a detachable member to the suture while the suture is housed within the shaft. The suture material may be any material as is known to one skilled in the art such as absorbable polyglycolic acid, polylactic acid, Monocryl and polydioxanone, as well as non-absorbable materials such as nylon, polyester, PVDF, or polypropylene. The suture may be a biodegradable polymer such as poly(lactic acid) (PLA), a polymer of glycolic acid (PGA), polycaprolactone (PCL), either braided or not braided. The suture may be a non-absorbable polymer such as Nylon or polypropylene, either braided or not braided. Alternatively, the suture may be a biologic (non-synthetic) material such as collagen or silk. In embodiments, the suture may be a metal, such as stainless steel, either braided or not braided. The suture may be any thread, braid, or monofilament that may be tied by knotting. The suture may be housed in a spool and may be capable of being cut into any arbitrary length for tying and knotting.

In embodiments, the detachable member is attached to a terminus of the suture and comprises an open loop as a detachable loop. The detachable loop may be any shaped loop including oblong, square, rounded, or triangular such that the needle can catch the loop upon exiting the shaft housing. The detachable member functions to connect to the suture, connect to the needle, and passively lock on the deflectable member. In embodiments, the detachable member may be made of stamped or molded or cast metal or plastic. The metal may be a shape-memory metal or super-elastic material such as described above. The plastic may be flexibly deformable. In embodiments, the detachable member is flexibly deformable to fit through the locking grid of the receiving area of the deflectable member. The detachable loop may comprise a flange or wings that flex as the detachable loop passes through the locking grid but expand to prevent the detachable loop from retracting back through the grid as the needle is retracted.

In embodiments, the device may include a deflectable member wherein the deflectable member provides a catch that is deflected away from the axis of the shaft. In this embodiment, the needle may be made of shape memory material and is held in a straight configuration when the needle is confined within the shaft. When the needle slides from the exit port out of the shaft, the needle assumes a curved shape to bring the hook of the needle and the detachable member to the catch on the receiving mechanism of the deflectable member. The detachable member fits into the receiving mechanism of the deflectable member such that it is caught and will not retract back out when the needle is withdrawn.

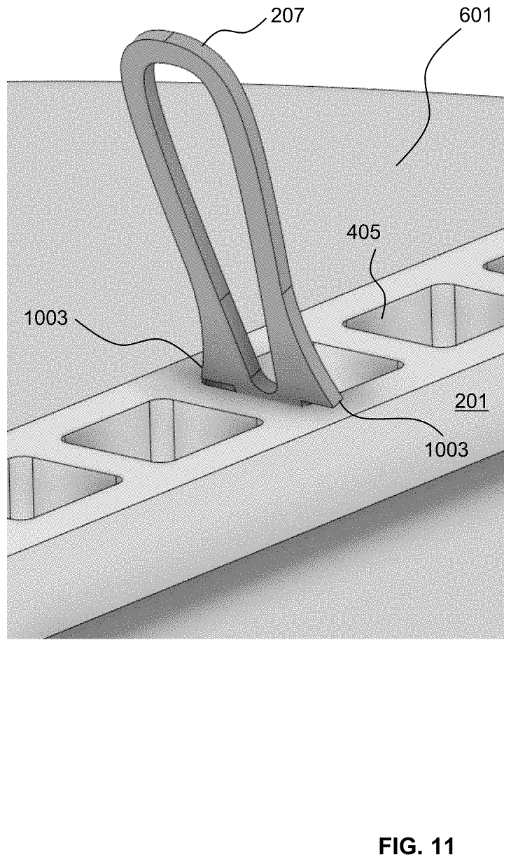

FIG. 11 illustrates an embodiment in which the detachable member is a detachable loop 207 locked into the receiving mechanism 405 of the deflectable member 201 as the deflectable member presents a tissue-facing surface on the surface of the tissue 601. The receiving mechanism in this embodiment comprises a plurality of catch positions to receive the detachable member upon exit from the tissue. The detachable loop 207 is shaped to flexibly deform to fit into the catch on the receiving mechanism and lock within the catch as the needle is retracted and the device pulled away. The plurality of catch positions on the receiving mechanism of the deflectable member may be a series of gridded ports with holes into which the needle can deliver the detachable member. In other embodiments, the catch positions may be hooks or barbs for receiving and locking the detachable member into the deflectable member. As illustrated, the detachable loop comprises ears 1003 shaped to allow for passive locking into a catch such that the detachable loop will not retract back through the hole.

The deflectable member may be capable of positioning along the axis of the shaft or substantially deflected therefrom. When the deflectable member is in the first position, the first position is along the axis of the shaft such that the deflectable member and the shaft are substantially disposed in a long, thin, configuration slidable through several centimeters of a cannula inserted through a minimally-invasive surgical incision.

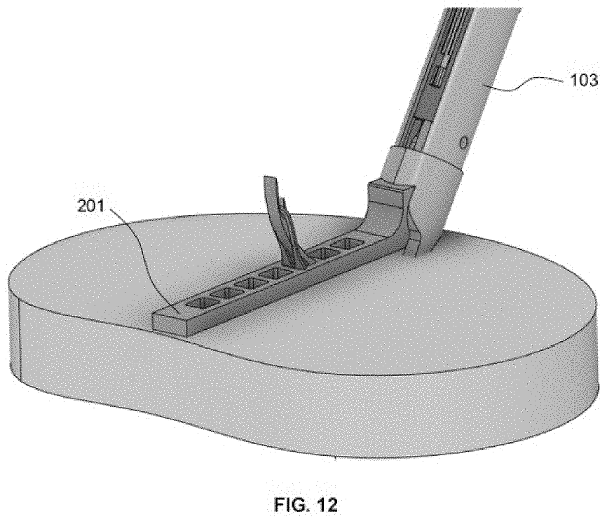

FIG. 12 illustrates an embodiment where, alternatively, the deflectable member 201 may be deflected away from the axis of the shaft 103 such that when the needle is advanced out of the shaft to form the suture into a stitch in the tissue, the catch on the receiving mechanism catches the detachable member at an angle offset from the axis of the shaft. In this embodiment, the first end of the stitch is made substantially along the axis of the shaft and the second end of the stitch is made at a position displaced radially from the axis of the shaft.

In embodiments, when the device is pulled away from the tissue, the detachable member remains connected to the catch of the deflectable member and is removed from the suture, leaving only the suture in the tissue. Further, the delivery needle may be retracted back into the shaft after the detachable member is caught by the catch on the deflectable member.

The suture may be delivered into the tissue and back out of the tissue at entry and exit points defining a stitch length wider than the diameter of the shaft. Alternatively, the length of the suture stitch may be wider than the diameter of the shaft and at least double the diameter of the shaft.

In embodiments, the catch is positioned on a bendable appendage to the shaft and the catch receives and catches the detachable member at a tissue exit point substantially spaced away from an axis of the shaft.

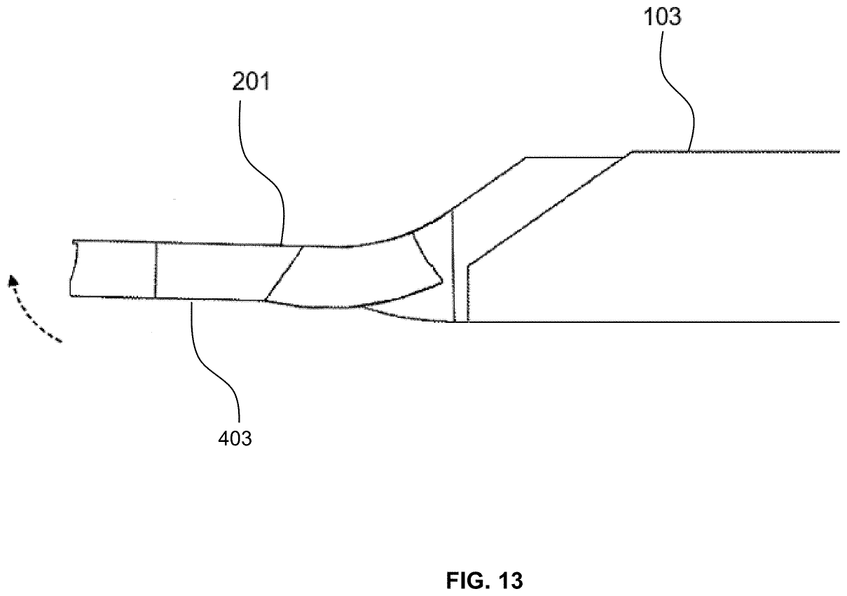

FIG. 13 illustrates the deflectable member in a first position extending along an axis of the shaft 103. The axis of the shaft is not necessary a tangible object and instead may be thought of as a reference line defining a geometric center of the substantially cylindrical shaft 103, useful to measure the angle of deflection by the deflectable member in relation to the fixed position of the shaft. The deflectable member presents a tissue-facing surface 403 against the tissue to be sutured.

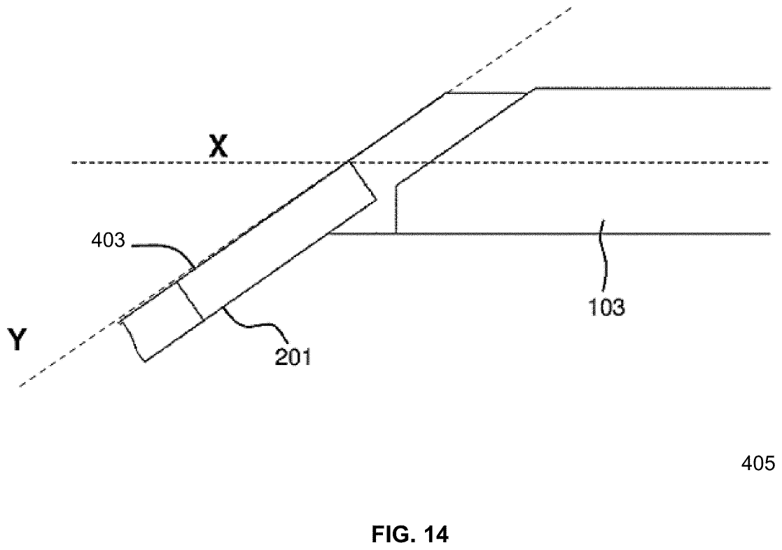

FIG. 14 shows the deflectable member 201 in a second position with a tissue-facing surface 403 extending along line Y. The second position of the deflectable member may be substantially offset from the axis of the shaft 103 such that line Y and the axis X of the shaft form an acute angle. However, the deflectable member may be deflected away from the axis of the shaft at any angle from 0 degrees to 180 degrees. The deflectable member 201 may occupy the first position (extending along the axis X) or second position (deflected from the axis X). The deflectable member may be biased to preferentially occupy either the first position or the second position, with the ability to be elastically deformed into the other of the first and second positions. In embodiments, the distal end of the shaft includes one or more pointed tips adapted to enable the surgeon to facilitate tissue approximation prior to suture deployment.

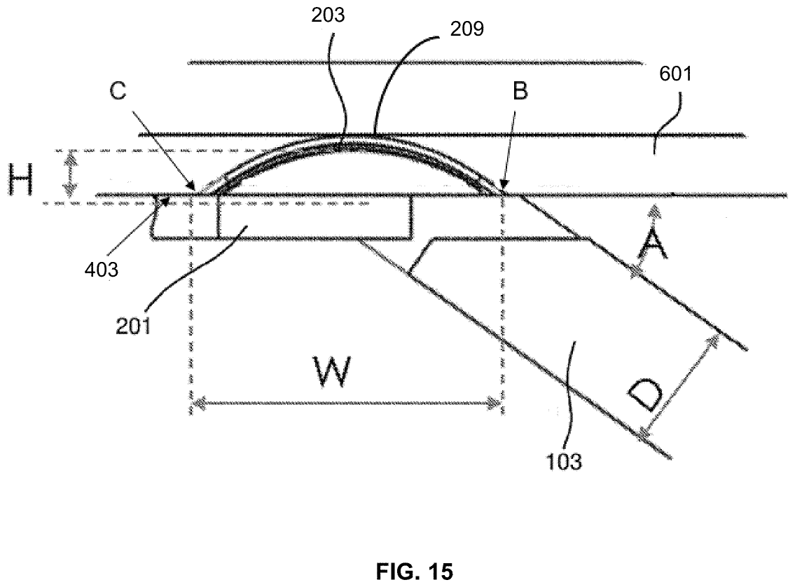

FIG. 15 shows the needle 203 carrying the suture 209 out of the exit port and entering into the tissue 601 at a first entry point 1507 along a curved path to a second exit point 1509 at the deflectable member 201. As shown, the device has been used to deliver a surgical suture with a defined stitch width W across a surface of the tissue that penetrates to a depth of H within the tissue. Because the deflectable member 201 is biased away from the shaft 103 such that the axis of the shaft 103 and the tissue-facing surface 405 form an acute angle, A, the shaft forms the angle A with the surface of the tissue 601. In preferred embodiments, the shaft is dimensioned for minimally-invasive surgery and has a length L of at least 15 cm and has a diameter D of less than 1 cm. More preferably, L≥25 cm and D≤7 mm. Most preferably, H<D<W.

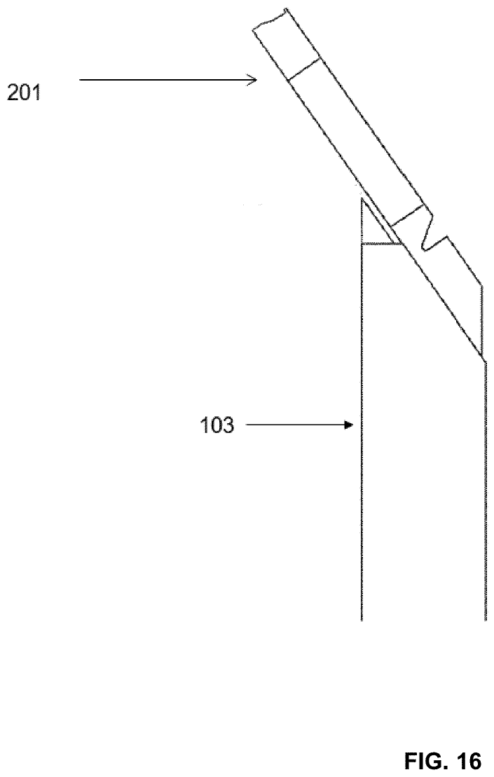

FIG. 16 illustrates an embodiment wherein the deflectable member 201 may be coupled to the shaft 103 by a hinge or pivot that allows the deflectable member to rotate relative to the fixed axis of the shaft. The hinge may be any suitable hinge that allows for deflection from at least a first position to a second position relative to the fixed axis of the shaft, for example, a flexibly deformable material, a notch, a curved wrist-joint, hinge pin or other joint.

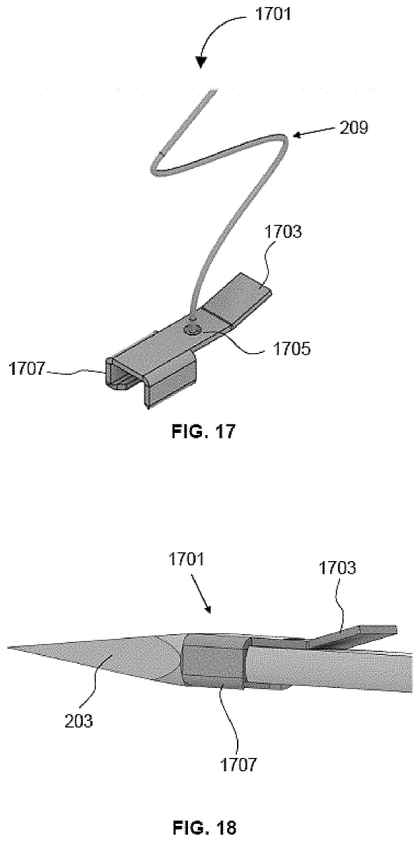

FIG. 17 illustrates an alternative embodiment wherein the detachable member is a detachable clip 1701. The detachable member functions as a suture connector in that it connects to the suture and to the needle to carry the suture in and out of tissue to deliver the suture to the deflectable member, thus serving as a temporary connector between the suture and the needle. In this design, the detachable member is a detachable clip 1701. The detachable clip may be a metal or polymer device and functions to connect to the curved needle, connect to the suture, and attach to the deflectable member. In embodiments, the detachable clip is flexibly deformable to fit within a grid of locking ports on the deflectable member. The detachable clip may comprise a tab 1703 angled upwards away from the horizontal axis of the clip. The tab may be a rigid or flexible piece such that the tab is capable of passively locking into the receiving mechanism of the deflectable member. The detachable clip as a whole may be a rigid or flexible metal or polymer device capable of connecting to a first terminus of the suture, connecting to the needle, and also capable of locking in the receiving mechanism on a deflectable member. The detachable clip may be connected to the suture by, for example, a hole 1705 or other mechanism.

The head end of the detachable clip, opposite the tab, includes two folded edges capable of connecting to the curved needle from the side by attaching the folded edges to the needle. One of the folded edges may include a second fold 1707 to create a curved bracket for snapping or clicking onto the curved needle such that the detachable clip is attached to the needle and is carried by the needle in and out of the tissue and delivered to the receiving mechanism of the deflectable member. In other embodiments, the detachable clip may be shaped to fit into a groove, notch, slot, or cut-out on the curved needle, for example by snapping into a corresponding fitted slot on the curved needle to provide a clasp or friction fit to the needle. The detachable clip may include a hole to which a terminus of the suture is attached.

FIG. 18 illustrates the detachable clip 1701 as it attaches to the needle 203 from the side by snapping the folded edges 1707 onto the needle. Once the folded edges of the detachable clip click or snap onto the needle, the detachable clip lies flat against the needle except for the tab 1703 biased to bend slightly upward from the axis of the needle. Once attached, and as described for the detachable loop 207 embodiment, the detachable clip 1701 fits into the receiving mechanism of the deflectable member such that it is caught and will not retract back out when the needle is withdrawn. Thereafter, when the needle retracts back into the shaft, the receiving mechanism of the deflectable member retains the detachable clip and detaches the detachable clip from the end of the suture. Further, when the device delivers the suture into tissue and the device is pulled away from the tissue, the deflectable member carries the detachable clip away from the tissue.

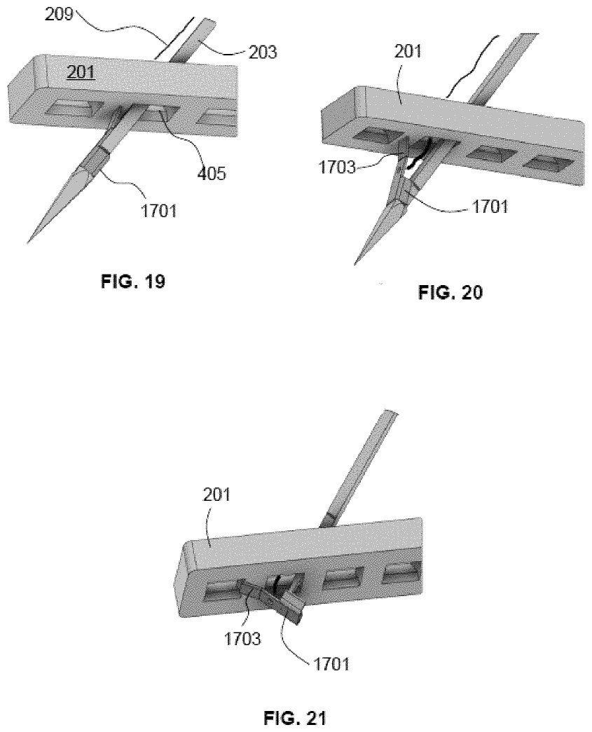

FIG. 19 shows the detachable member as a detachable clip 1701 coupled to the suture 209 and the curved needle 203. The detachable clip is clipped to the needle and pulled by the needle to the receiving mechanism 405 on the deflectable member 201. In this embodiment, the receiving mechanism is a series of square holes in the shelf of the deflectable member. When the needle carries the first end of the suture with the detachable member to the receiving mechanism on the deflectable member, with the deflectable member spaced away from the axis of the shaft, the receiving mechanism of the deflectable member captures the detachable clip. The detachable clip peels off from the needle after passing inside one of the square holes on the deflectable member. Thereafter, when the needle retracts back into the shaft, the receiving mechanism of the deflectable member retains the detachable clip and detaches the detachable clip from the first end of the suture.

FIG. 20 shows the detachable clip 1701 being prevented from moving back though the square holes by the geometric constraints of the device, for example by the tab 1703 which passively locks to the receiving mechanism of the deflectable member 201.

FIG. 21 shows the detachable clip 1701 captured by the deflectable member 201. As shown, when the device delivers the suture into tissue and the device is pulled away from the tissue, the deflectable member 201 carries the detachable clip 1701 away from the tissue. In embodiments, the detachable clip is flexibly deformable to fit within a grid of locking ports on the deflectable member 201. The shape of the detachable clip thereafter prevents the detachable clip from travelling back through the locking port of the deflectable member as the needle is retracted thus locking the detachable clip within the receiving mechanism of the deflectable member as the device is pulled away from the tissue. In embodiments, when the device is pulled out from the patient's body, the end of the suture that passed though the tissue is passively locked on the deflectable member.

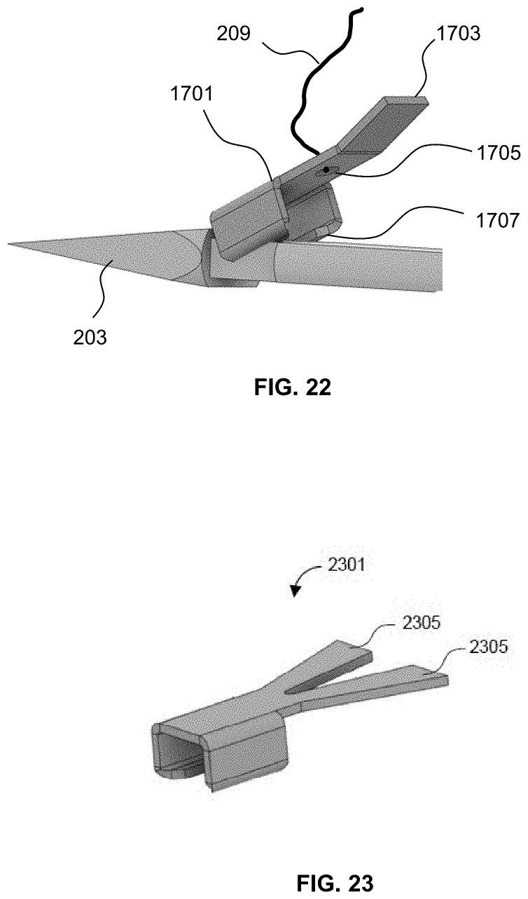

FIG. 22 illustrates an embodiment of the detachable member as a detachable clip 1701 coupled to the suture 209. The detachable clip includes the tab 1703 biased in an upwards direction for catching onto the receiving mechanism of the deflectable member. The detachable clip may be connected to the suture at a hole 1705 mechanism. The folded edge 1707 creates a curved bracket for snapping or clicking onto a notch of the curved needle 203 such that the detachable clip is attached to the needle and lies flat against the needle with the exception of the slightly biased upward tab. The detachable clip may be rigid or flexibly deformable in order for the needle to push the detachable member through a hole of the locking grid on the receiving mechanism.

FIG. 23 illustrates a further embodiment of the detachable member designed as a y-shaped member 2301. The y-shaped member 2301 may have wings 2305 that are flexibly deformable to compress or fold over each other to pass through the receiving mechanism of the deflectable member. The head end of the y-shaped member may include two folded edges capable of connecting to the curved needle from the side by attaching the folded edges to the needle. One of the folded edges may include a second fold to create a curved bracket for snapping or clicking onto the curved needle such that the y-shaped member is attached to the needle and is carried by the needle in and out of the tissue and delivered to the receiving mechanism of the deflectable member. In other embodiments, the y-shaped member may be shaped to fit into a groove, notch, slot, or cut-out on the curved needle, for example by snapping into a corresponding fitted slot on the curved needle to provide a clasp or friction fit to the needle.

As with the detachable clip 1701 and detachable loop 207 embodiments of the detachable member described previously, the y-shaped member 2301 may attach to a terminus of the suture via a hole or other connection mechanism such as a v-slot, catch, clasp, shackle, ring, or hook. The y-shaped detachable member may be caught by or attached to the needle and carried to the receiving mechanism on the deflectable member. The y-shaped member may include compressible wings biased outward that can be pushed together to pass through the receiving mechanism of the deflectable member. Thereafter, the wings expand to lock the y-shaped member into the receiving mechanism. When the needle retracts back into the shaft, the receiving mechanism of the deflectable member retains the y-shaped member and detaches the y-shaped member from the end of the suture. Further, when the device delivers the suture into tissue and the device is pulled away from the tissue, the deflectable member carries the y-shaped member away from the tissue.

In embodiments, the y-shaped member may be rigid or flexibly deformable to fit within a grid of locking ports on the deflectable member. The y-shaped member may be shaped to fit into a groove, notch, slot, catch, or cut-out on the curved needle, for example by snapping into the corresponding slot on the curved needle.

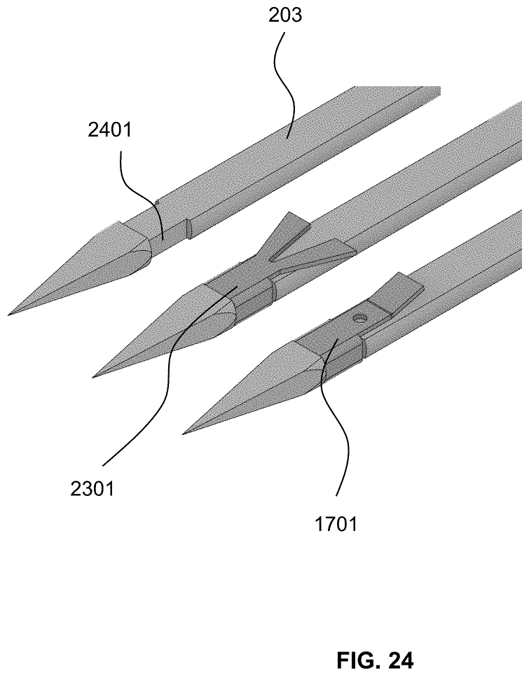

FIG. 24 illustrates an embodiment of the curved needle 203 that includes one or more notches 2401 fitted for the detachable member to clip or slide into such that the detachable member is held by the needle. The notch may also be in the form of a cut-out, groove, slotted hole, notch, or fitted slot. The y-shaped member 2301 is shown attached at the notched portion of the curved needle for the needle to carry the detachable member and suture into tissue at a first entry point and out of tissue at a second exit point to the receiving mechanism on the deflectable member. The y-shaped member may have wings or fins that are rigid or flexibly deformable and operate to fit within a grid of locking ports on the deflectable member such that the y-shaped member is locked into the grid as the needle is retracted. The detachable clip 1701 is also illustrated as clipped onto the needle for delivery into and out of the tissue to the deflectable member.

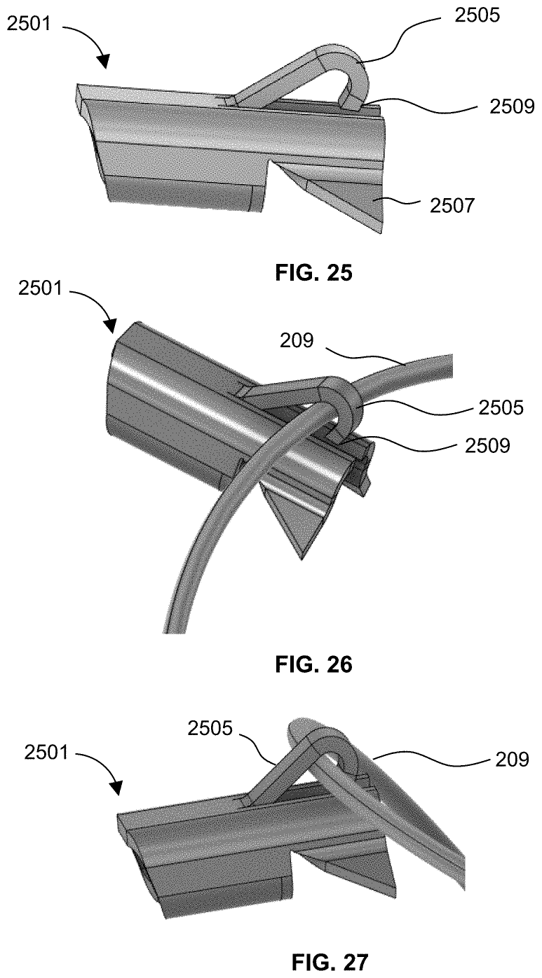

FIG. 25 illustrates a further embodiment of the detachable member designed as a winged connector 2501 wherein the detachable member has a coupling loop 2505 or coupling link to attach to or hook the suture. The coupling loop may be plastic or metal loop with a spring-loaded gate for connecting the suture to the detachable member. In other embodiments, the coupling loop may be a loop comprising a gap 2509, wherein the loop is biased to a position pressed against the surface of the detachable member such that the gap is closed. In this embodiment, suture may be connected to the coupling loop by threading the suture through the loop. The detachable member may then be removed from the suture by pressure that opens the gap. Alternatively, the suture may comprise a loop on a terminus that may be connected to the coupling link on the detachable member by passing the loop through the gap on the detachable member.

In embodiments, the detachable member may have one or more tapered wings 2507 at one end that flare laterally from the end of the detachable member along a longitudinal line from the center of the detachable member. The wings may be flexibly deformable to compress or fold to pass through the receiving mechanism of the deflectable member. As with other embodiments of the detachable member, the head end of the winged connector may include two folded edges capable of connecting to the curved needle from the top or side of the needle by attaching the folded edges to the needle. One of the folded edges may include a second fold to create a curved bracket for snapping or clicking onto the curved needle such that the detachable member is attached to the needle and is carried by the needle in and out of the tissue and delivered to the receiving mechanism of the deflectable member. In other embodiments, the detachable member may be shaped to fit into a groove, notch, slot, or cut-out on the curved needle, for example by snapping into a corresponding fitted slot on the curved needle to provide a clasp or friction fit to the needle.

FIG. 26 illustrates an embodiment of the detachable member as a winged connector 2501 wherein suture 209 has been threaded into the coupling loop 2505. The suture may be threaded through the coupling loop, or, alternatively, the suture may be pressed against the gap 2509 of the coupling loop such that it passes through the gap and is caught within the coupling loop.

A detachable loop or detachable member of the disclosure may be adapted or configured for quick connection to a suture during surgery, e.g. the suture snap mechanism using the coupling loop 2505. The detachable member may include the coupling loop 2505 as a snap mechanism allowing for connection of the suture to the detachable member manually, while using the device. The snap mechanism may include a coupling loop 2505, wherein the suture may be pressed against a gap of the coupling loop such that the suture passes through the gap and is caught within the coupling loop.

FIG. 27 illustrates the detachable member as a winged connector 2501 with a coupling loop 2505 wherein a loop of suture 209 has been pressed through the gap of the coupling loop. Once the loop of suture is within the coupling loop, it is retained within the coupling loop by the shape of the loop and its bias to a closed position against the surface of the detachable member.

As with the detachable clip 1701, the detachable loop 207, and the y-shaped member 2301 embodiments of the detachable member described previously, the detachable member designed as a winged connector may also attach to a ring, hook, loop or other catch attached to a terminus of the suture. The winged connector detachable member may be caught by or attached to the needle and carried to the receiving mechanism on the deflectable member. The wings of the winged connector may be any shape that flares from the body of the detachable member. The wings of the detachable member may be biased outward and compressible or flexibly deformable such that the detachable member can pass through the receiving mechanism of the deflectable member. Thereafter, the wings expand to lock the winged connector member into the receiving mechanism. When the needle retracts back into the shaft, the receiving mechanism of the deflectable member retains the winged connector and detaches the winged connector from the suture. Further, when the device delivers the suture into tissue and the device is pulled away from the tissue, the deflectable member carries the winged connector detachable member away from the tissue.

In embodiments, the winged connector may be rigid or flexibly deformable to fit within a grid of locking ports on the deflectable member. The winged connector may be shaped to fit into a groove, notch, slot, catch, or cut-out on the curved needle, for example by snapping into the corresponding slot on the curved needle.

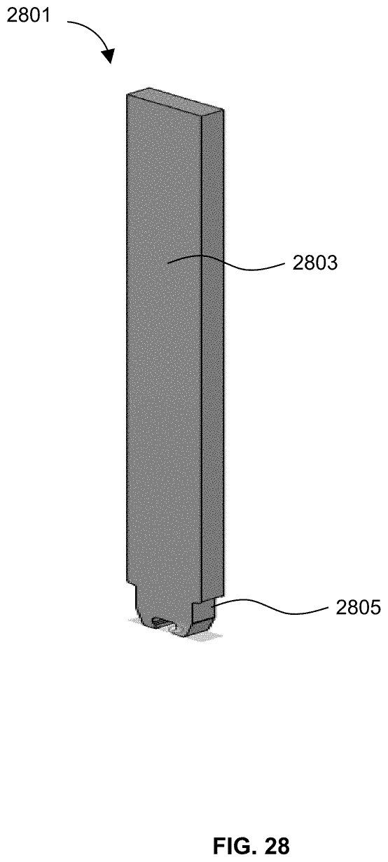

FIG. 28 illustrates an embodiment of the invention that includes a connector device 2801 for attaching the detachable member to the needle. In embodiments, the connector device includes a rod 2803 on the proximal end and connecting head 2805 on the distal end. The connecting head may have grooves or ridges with a complementary shape to connect to a detachable member. In embodiments, the connector device is made of any suitable material such as plastic or metal such that pressure from the proximal end of the rod applied in a downward position forces the connecting head to expand to attach to the detachable member. In embodiments, the attachment mechanism is via the grooves, ridges or complementary shape of the connecting head. Alternatively, the connecting mechanism may be a magnetic connector or other coupler. In other embodiments, the connecting head of the connector device may have jaws or a mouth that is flexibly deformable such that the jaws or mouth are biased inward but flex outward with applied pressure to clamp onto or attach to the detachable member to hold it for attaching to the needle. The connector device 2801 may be used as a suture attachment rod to assist with the connection of the detachable loop to the insertion needle during surgery.

Embodiments of the disclosure include kits. A kit of the disclosure may include a suturing device, at least one of these suture attachment rods (e.g., connector device 2801), and one or more detachable loop or detachable member. In preferred embodiments, each detachable loop or detachable member is pre-attached to one suture attachment rod or connector device 2801. The device may be provided in a kit that includes a connector device 2801 for attaching the detachable loop to the suture thread. The connector device 2801 may include an extended body with a connecting head at one end. The connecting head may include grooves or ridges with a shape complementary to the detachable loop. Optionally the kit includes one or more of the connector devices wherein each connector device is pre-attached to a respective detachable loop.

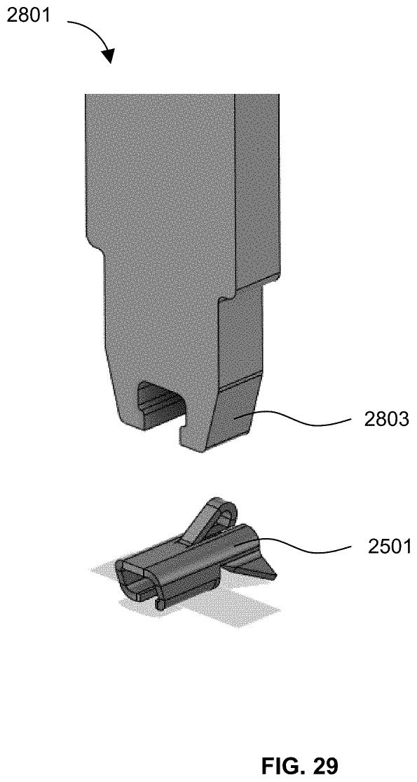

FIG. 29 illustrates an embodiment of a connector device 2801 with connecting head 2805 shaped to fit around the body of a detachable member shaped as a winged connector 2501. In this embodiment, the grooves, jaw, or ridges of the connecting head are designed to flex outward when the connecting device is pushed downward onto the detachable member such that the connecting head clips onto and holds the detachable member within the connecting head. The mouth or jaws of the connecting head is biased to an inward position and therefore holds the detachable member through the pressure of the connecting head mouth retracting from the flexed position back to the biased inward position.

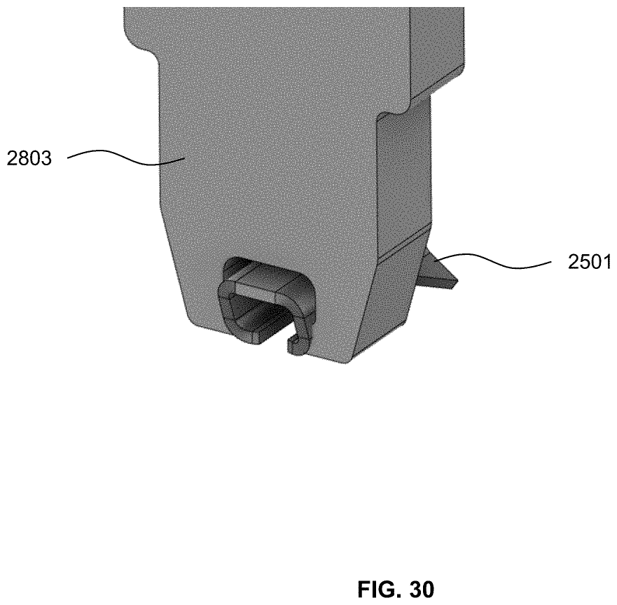

FIG. 30 further illustrates an embodiment of the connector device 2801 attached to the detachable member with the detachable member held within the connecting head.

The invention includes a method for connecting the suture to the detachable member and loading the detachable member onto the needle housed within the device. Alternatively, the invention provides a method of connecting the detachable member to the needle wherein the suture is then caught by the needle.

In embodiments, the methods of the invention include providing a suturing device according to embodiments of the disclosure, having a needle delivery slot housed within the distal end of the shaft and opening to the exit port, wherein the needle may be held within the distal end of the shaft at the exit port and may be operably coupled to the distal end of a driver member disposed within the shaft. The driver member may be actuated to push the needle with the detachable member and suture out of the shaft and into the tissue. Thus, the needle delivery slot allows the driver member to push the needle through the exit port and into the surface of the tissue at a first entry point and out of the tissue at a second exit port. The method includes providing a connector device according to embodiments of the invention; connecting the connecting head of the connector device to the detachable member; inserting the detachable member into the shaft of the device; attaching the detachable member onto the needle; and retracting the connector device from the detachable member. In embodiments, the method includes the connector device connecting both the suture to the detachable member and the detachable member to the needle. In other embodiments, the method includes the connector member connecting the detachable member to the suture and to the needle, and loading the needle with the detachable member and suture into the shaft of the device.

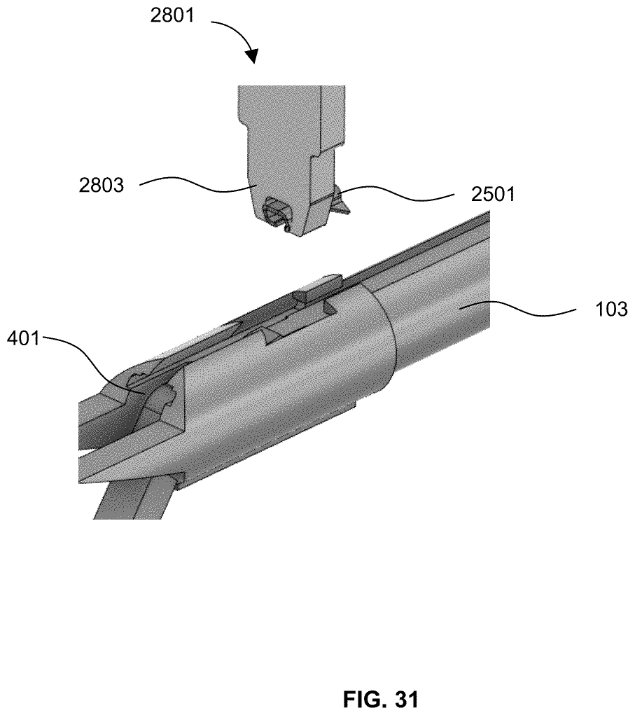

FIG. 31 illustrates an embodiment in which the connector device 2801 holds a detachable member shaped as a winged connector 2501 in the mouth of the connecting head 2805, wherein the connector device will deliver the detachable member to the shaft 103. In embodiments, the detachable member is connected directly to the needle housed within the shaft. In other embodiments, the detachable member is delivered to the housing within the shaft where the needle will attach to the detachable member upon exit from the exit port 401. The

In embodiments, the suture is directly connected to the detachable member by the user. Alternatively, the suture is placed within the housing of the shaft to be caught by the detachable member or by the needle as the needle is deployed out of the housing. In still another embodiment, the suture may comprise a cartridge of suture with a loop on a terminus of the suture pre-loaded and automatically connected to the needle within the housing for example by a dedicated magazine mechanism.

As previously described, the suture may be coupled to a detachable member such as a detachable loop which locks into the receiving mechanism of the deflectable member. In some embodiments, the devices of the invention include a spring-loaded mechanism in the handle in order to facilitate faster application which may be necessary for better penetration through tissue. The movement of the needle along the path may be made possible by the action of the driver member. The driver member may retract the needle back into the shaft once the suture has been received by the receiving mechanism of the deflectable member. Thus, in some embodiments, the operation of the trigger causes the driver member to push the needle with an end of suture attached to the needle, out of the exit port and into the tissue at a first entry point along a path in alignment with the axis of the deflectable member. The needle then exits the tissue at a second exit point to deliver the suture to the receiving mechanism of the deflectable member. Once the deflectable member has received the suture, the needle is retracted back into the shaft by operation of the driver member leaving the ends of the suture suture extending from the tissue surface at the entry and exit points.

Additional embodiments of the invention may be related to an insertion device for inserting suture into a guide slot for feeding to the needle end. In embodiments, devices of the invention provide for the needle to catch the end of the suture and carry the suture along a curved path into the tissue at a first entry point and out of the tissue at a second exit point to deliver the suture to the receiving mechanism on the deflectable member.

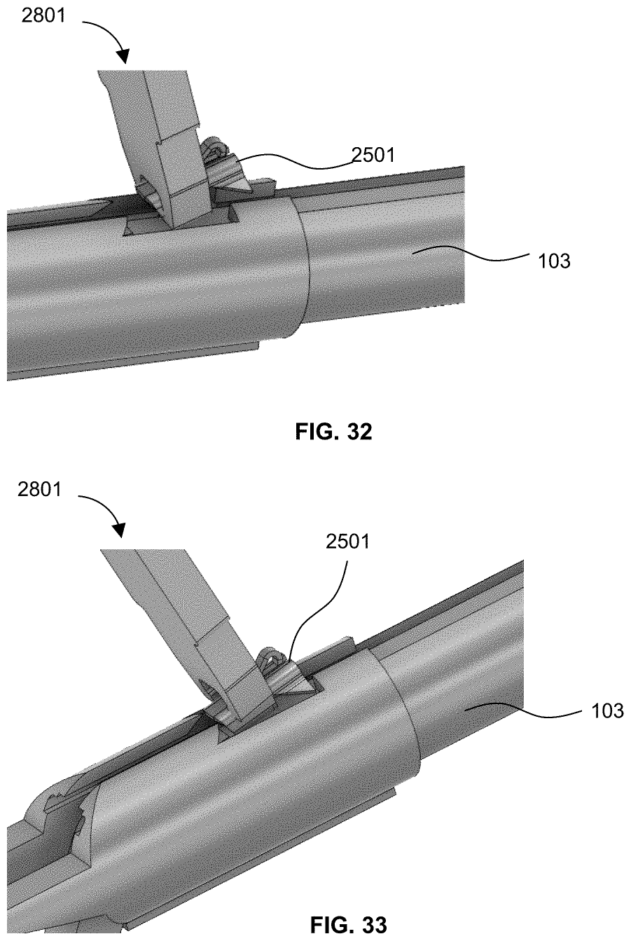

FIG. 32 further illustrates the action of the connector device 2801, attached to the detachable member shaped as a winged connector 2501, inserting the detachable member into the shaft 103 housing to connect to the needle therein. In embodiments the connector device holds the detachable member via the pressure exerted by the mouth of the connecting head retracting from the flexed outward position to the biased inward position.

FIG. 33 illustrates an embodiment of the method of connecting the detachable member to the needle wherein the connection device 2801, with the detachable member shaped as a winged connector 2501 attached to the connecting head 2805, is inserted into the shaft at an angle to connect to the needle housed near the exit port of the shaft.

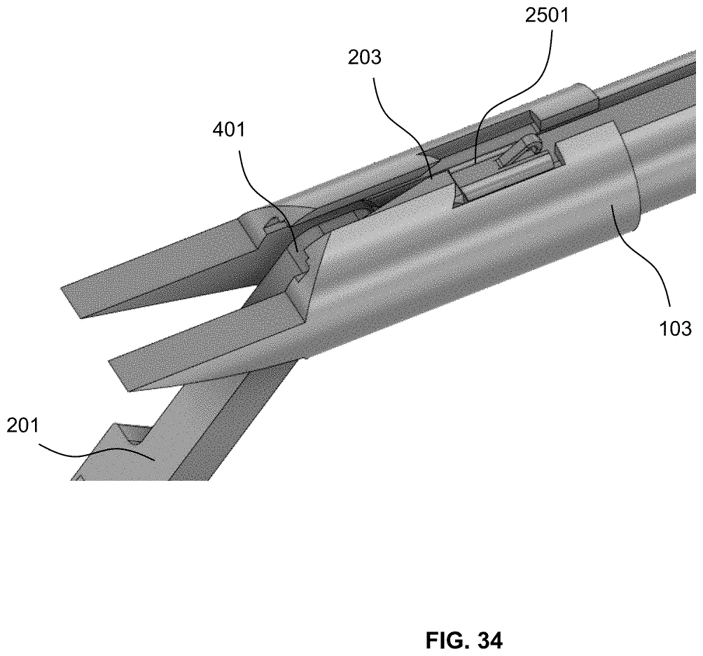

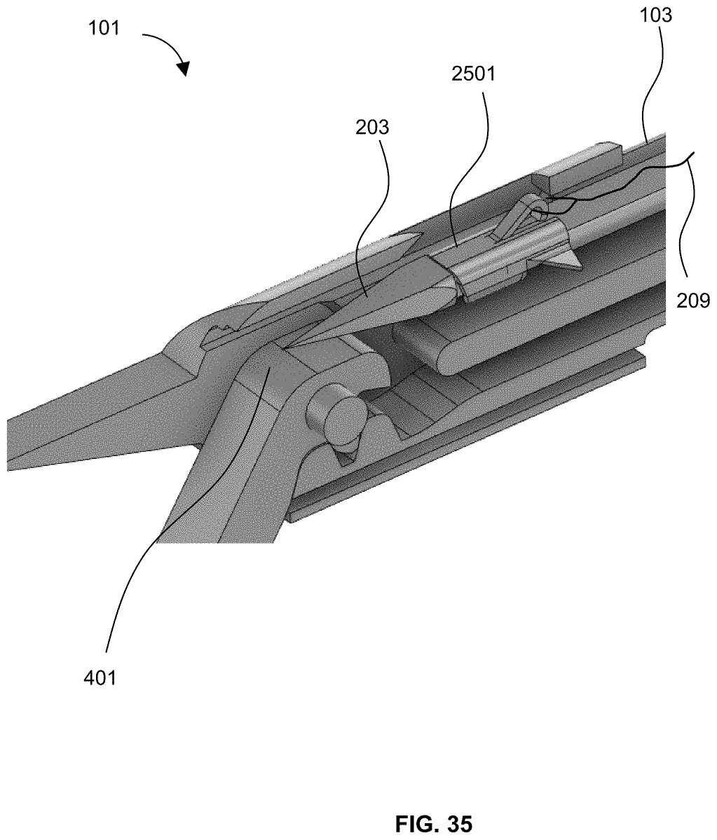

FIG. 34 illustrates the needle 203 with detachable member shaped as a winged connector 2501 attached. The needle is housed in the shaft 103, near the exit port 401. As discussed previously, in embodiments, the device may include a needle delivery slot housed within the distal end of the shaft and opening to the exit port 401. The needle may be held within the distal end of the shaft 103 at the exit port 401 and may be operably coupled to the distal end of a driver member disposed within the shaft. The driver member may be actuated to push the needle with the suture and the detachable member out of the shaft and into the tissue. Thus, the needle delivery slot allows the driver member to push the needle through the exit port 401 and into the surface of the tissue at a first entry point and out of the tissue at a second exit port to the receiving mechanism of the deflectable member 201. In some embodiments, the devices of the invention include a spring-loaded mechanism in the handle in order to facilitate faster application which may be necessary for better penetration through tissue.

FIG. 35 illustrates a cross-section view of an embodiment of the device of the invention in which the needle 203, with a detachable member shaped as a winged connector 2501 attached to the needle, is housed within the shaft 103 of the device, near the exit port 401. In this view the detachable member is connected to both the needle and the suture 209.

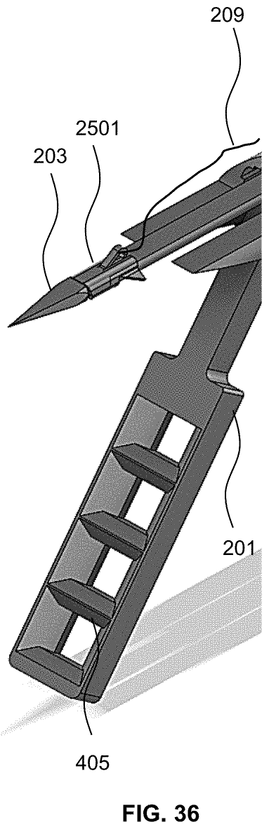

FIG. 36 illustrates the needle 203, with the detachable member shaped as a winged connector 2501 connected to both the needle and the suture 209, exiting the shaft of the device. The figure illustrates an embodiment of the deflectable member 201 and the receiving mechanism 405 of the deflectable member, to which the needle delivers the suture and the detachable member.

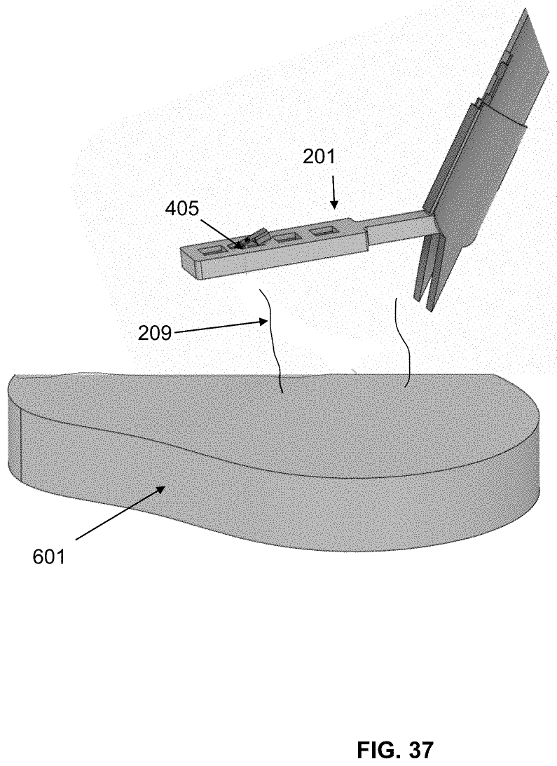

FIG. 37 illustrates another embodiment in which the receiving mechanism 405 of the deflectable member 201 receives the suture 209 after the needle exits the tissue and delivers the suture to the receiving mechanism on the deflectable member. Embodiments of the detachable member lock into the receiving mechanism grid such that the detachable member stays in the receiving mechanism as the needle is retracted. The locking may be due to the geometric constraints of the size and shape of the detachable member in relation to the holes or grid on the receiving mechanism. The detachable member may be passively locked into the receiving mechanism of the deflectable member by the geometric constraints of the size and shape of the member, or, locking may be achieved by a snap, clip, catch, or fastener designed to lock the detachable member into the receiving mechanism of the deflectable member.

The detachable member is retained in the receiving mechanism as the device is pulled away from the tissue 601. In embodiments, the detachable member is peeled off from the needle after passing inside one of the locking grids on the deflectable member. In some embodiments, the locking grids may be square or rounded holes. Therefore, the detachable member is prevented from moving back through the locking grid because of the geometric configuration of the detachable member. The detachable member remains passively locked to the deflectable member as the device is removed from the suture site.

In embodiments, devices of the invention comprise a body with a handle, an elongated shaft extending from the body, a deflectable member coupled to the distal end of the shaft, a needle housed within the shaft, and a suture attached on the needle. The deflectable member is moveable between at least a first position extending along an axis of the shaft and a second position deflected away from the axis of the shaft. The needle housed within the shaft is in a substantially straight configuration and comprises a shape memory material that, when the needle is advanced out of the shaft with the deflectable member in the second position, gives the needle a curved shape that carries the tip of the needle to a location on the deflectable member spaced away from the axis of the shaft. The tip of the needle carries a first end of the suture on the needle to a location on the deflectable member spaced away from the axis of the shaft. Various embodiments also provide a detachable member shaped as a loop, hook, clip, tab, catch, or loop piece that attaches to an end of a suture and is used by the suturing device to catch and hold the distal end of the suture in a position while the delivery mechanism retracts, in which, when the device is pulled away, the detachable loop is detached from the suture and carried away with the device leaving only the suture extending through the tissue. Further, embodiments include a connecting device for connecting the suture with the detachable member and for connecting the detachable member to the needle. In other embodiments, the shaft is designed to house the detachable member within the shaft for connection to the needle as the needle exits the shaft.

The invention provides methods for attaching the suture to the detachable member, the detachable member to the needle and or the suture to the needle. Further method embodiments include loading the detachable member, with or without the suture attached, into the device for connection to the needle

Devices of the invention may be used to suture any type of anatomical tissue in any type of anatomical cavity for any type of anatomical surgery such as organ or tissue repair. The tissue may be soft or hard tissue. In some embodiments, the devices may be used to suture, ligate, and fixate tissue or an implant such as a mesh. The devices may be used for repair of tissue defects, anastomosis of vessels or organs, suturing of access sites, anchoring or fixating soft or hard tissue, fixating meshes or other implants or devices to a tissue, closing natural or artificial tissue openings, modifying anatomical spaces, lifting, stretching, pulling or retracting tissues or organs using open or minimally invasive approaches.

The devices may be used for open surgery or minimally invasive surgeries, including laparoscopic or endoscopic surgeries, and may be used with catheters and other small and large diameter tubular or hollow, cylindrical members providing access to small cavities such as veins or arteries, as well as large cavities such as the abdomen. The device is capable of adapting to and adjusting to different tissue surfaces and incision sites for a variety of stitch widths to meet surgical suturing needs. The device can be used in many surgical environments, including, for example, open, mini-open, and endoscopic, and with visualization, limited visualization, or no visualization of the suture delivery.

For example, and not intended to be limiting, the device may be use for laparoscopic, endoscopic or open surgical procedures such as orthopedic repair procedures to pass suture through tissue to bring the tissue in apposition with a bone, meniscus repair of a knee, to ligate, join, re-attach tendon to bone or otherwise secure adjoining tissue, or to secure a catheter or wound. Additionally, devices of the invention may be used for any procedure requiring the medical provider to operate remotely by manipulating instruments through small punctures or through natural orifices such as the vagina, anus, or esophagus. The device may also be positioned in a femoral artery and may be used for suturing body lumens to allow for suturing of vascular puncture sites located at the distal end of a percutaneous tract. The devices may be used for closure of wounds, closure of fascia, hemostasis, intestinal anastomosis and enterotomy, urogenital tract surgery, surgery of the musculoskeletal system, sinus surgery, vascular surgery, ocular surgery, plastic surgery, and neurosurgery as examples.

As used here and throughout the disclosure, “suture”, “thread”, and “suture thread” includes thread-based, braided, not braided, or monofilament sutures. The sutures may be any thread or string that may come from a spool, can be tied, and can be knotted. The sutures can be cut to any arbitrary length and can be tied by knotting. The suture material may be any material as is known to one skilled in the art such as absorbable polyglycolic acid, polylactic acid, Monocryl and polydioxanone, as well as non-absorbable materials such as nylon, polyester, PVDF, or polypropylene. The suture may be a biodegradable polymer such as poly(lactic acid) (PLA), a polymer of glycolic acid (PGA), polycaprolactone (PCL), either braided or not braided. The suture may be a non-absorbable polymer such as Nylon or polypropylene, either braided or not braided. Alternatively, the suture may be a biologic (non-synthetic) material such as collagen or silk. In embodiments, the suture may be a metal, such as stainless steel, either braided or not braided. The suture may be any thread, braid, or monofilament that may be tied by knotting. The suture may be housed in a spool and may be capable of being cut into any arbitrary length for tying and knotting.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A suturing device comprising:
a body comprising a handle;
an elongated shaft extending from the body;
a deflectable member coupled to a distal portion of the shaft and moveable between at least a first position extending along an axis of the shaft and a second position deflected away from the axis of the shaft;
a needle housed within the shaft in a substantially straight configuration, the needle comprising a super-elastic material that, when the needle is advanced out of the shaft with the deflectable member in the second position, gives the needle a curved shape that carries a tip of the needle to a location on the deflectable member spaced away from the axis of the shaft; and
a suture thread on the needle, wherein the tip of the needle carries a first end of the suture thread to the location on the deflectable member spaced away from the axis of the shaft.

2. The device of claim 1, wherein the deflectable member comprises a tissue-facing surface, wherein the deflectable member is elastically biased to the first position but, when the device is pushed against the surface of a tissue, the deflectable member deflects to the second position with the tissue-facing surface facing the surface of the tissue.

3. The device of claim 1, wherein the device delivers the suture thread into and back out of tissue and leaves the suture thread extending within the tissue with the first end of the suture thread emerging from the tissue at an exit point and a second end of the suture thread emerging from the tissue at an entry point.

4. The device of claim 3, wherein the entry point and the exit point define a stitch length and the stitch length is substantially larger than a diameter of the shaft.

5. The device of claim 1, wherein the deflectable member receives the first end of the suture thread from the needle and holds the first end of the suture thread when the needle retracts into the shaft.

6. The device of claim 1, wherein the needle passes the suture thread through tissue and leaves the suture thread in the tissue with the first and the second ends of the suture thread extending out of the tissue at locations spaced apart a length on a surface of the tissue.

7. The device of claim 1, wherein the first end of the suture thread comprises a loop or crook that is caught by the deflectable member when the needle is advanced out of the shaft.

8. The device of claim 1, wherein the deflectable member catches the first end of the suture thread when the needle is advanced out of the shaft to form the suture thread into a stitch in the tissue, wherein a first end of the stitch is initially made substantially along the axis of the shaft, and a second end of the stitch is initially made substantially displaced radially from the axis of the shaft.

9. The device of claim 1, wherein when the deflectable member is in the first position, the shaft and the deflectable member are substantially disposed in a long, thin configuration slidable through several centimeters of a cannula inserted through a minimally-invasive surgical incision.

10. The device of claim 1, wherein the tip of the needle comprises a sharpened point that pierces through tissue when the needle is advanced into tissue.

11. The device of claim 10, wherein the needle comprises a hook that carries the first end of the suture thread.

12. The device of claim 11, wherein the first end of the suture thread is attached to a detachable loop that is pulled by the hook.

13. The device of claim 12, wherein when the needle carries the first end of the suture thread to the location on the deflectable member spaced away from the axis of the shaft, the deflectable member captures the detachable loop, and when the needle retracts into the shaft the deflectable member retains the detachable loop and detaches the detachable loop from the first end of the suture thread.

14. The device of claim 13, wherein the detachable loop is shaped to pass through an opening on the deflectable member and not retract back through the opening.

15. The device of claim 13, wherein when the device delivers the suture thread into tissue and is pulled away from the tissue, the deflectable member carries the detached detachable loop away from the tissue.

16. The device of claim 12, wherein the detachable loop is attached to the suture thread at the time of surgery.

17. The device of claim 1, wherein the deflectable member comprises a grid of catching ports spaced at regular intervals.

18. The device of claim 1, wherein a distal end of the elongated shaft includes one or more pointed tips extending from the distal end.

19. The device of claim 12, wherein the device is provided in a kit that includes a connector device for attaching the detachable loop to the suture thread.

20. The device of claim 19, wherein the connector device comprises an extended body with a connecting head at one end.

21. The device of claim 20, wherein the connecting head includes grooves or ridges with a shape complementary to the detachable loop.

22. The device of claim 19, wherein the kit includes one or more of the connector devices wherein each connector device is pre-attached to a respective detachable loop.

23. A suture delivery device comprising:
a body comprising a handle;
an elongated shaft extending from the body;
a suture thread within the shaft, wherein the suture thread comprises a detachable member attached to a first end of the suture thread;
a needle disposed within the shaft and slidable out of the shaft through target tissue, the needle comprising a hook engaged with the detachable member to pull the suture thread into and through the tissue; and
a catch on a portion of the shaft, wherein when the needle slides through the tissue, the hook emerges from the tissue and engages the detachable member with the catch,
wherein when the suture thread is within the tissue and the shaft is pulled away from the tissue, the catch pulls the detachable member off of the suture thread and away from the tissue, leaving only the suture thread within the tissue.

24. The device of claim 23, wherein the detachable member is attached to a terminus of the suture thread and comprises an open loop.

25. The device of claim 23, wherein the suture thread is made of braided or not braided synthetic polymer fibers, biodegradable polymer, non-absorbable polymer, a non-synthetic biologic material, or a metal, and the detachable member is made of stamped or molded or cast metal or plastic.

26. The device of claim 23, wherein the catch is provided by a deflectable member that can be deflected away from an axis of the shaft, and wherein the needle comprises a super-elastic material that is held in a straight configuration when the needle is confined within the shaft and, when the needle slides out of the shaft assumes a curved shape to bring the hook and detachable member to the catch on the deflectable member.

27. The device of claim 26, wherein the deflectable member comprises a plurality of catch positions to receive the detachable member upon exit from the tissue.

28. The device of claim 26, wherein when the deflectable member can be positioned along an axis of the shaft with the shaft and the deflectable member substantially disposed in a long, thin configuration slidable through several centimeters of a cannula inserted through a minimally-invasive surgical incision.

29. The device of claim 26, wherein when the device is pulled away from the tissue, the detachable member remains connected to the catch on the deflectable member and is removed from the suture thread, leaving only the suture thread in the tissue.

30. The device of claim 26, wherein the catch catches the detachable member when the deflectable member is deflected away from an axis of the shaft and the needle is advanced out of the shaft to form the suture thread into a stitch in the tissue, wherein a first end of the stitch is made substantially along the axis of the shaft, and a second end of the stitch is made at a position displaced radially from the axis of the shaft.

31. The device of claim 23, wherein the delivery needle comprises a pointed tip with the hook just proximal to the pointed tip along the delivery needle.

32. The device of claim 23, wherein the delivery needle is retracted back into the shaft after the detachable member is caught by the catch.

33. The device of claim 23, wherein the suture thread is delivered into the tissue and back out of the tissue at an entry point and an exit point defining a stitch length wider than a diameter of the shaft.

34. The device of claim 23, wherein the catch is positioned on a bendable appendage to the shaft and wherein the catch receives and catches the detachable member at a tissue exit point substantially spaced away from an axis of the shaft.

35. The device of claim 23, wherein a length of the suture stitch can be more than at least double a diameter of the shaft.

36. The device of claim 23, wherein the device passes the suture thread through tissue, removes the detachable member from the suture thread, and leaves only the suture thread in the tissue with a first and a second end of the suture thread extending out of the tissue at respective first and second exit locations spaced apart a length on a surface of the tissue.

37. The device of claim 23, wherein the detachable member comprises a snap mechanism allowing for connection of the suture to the detachable member manually, while using the device.

38. The device of claim 37, wherein the snap mechanism comprises a coupling loop, wherein the suture may be pressed against a gap of the coupling loop such that the suture passes through the gap and is caught within the coupling loop.
